(19) United States
(12) Patent Application Publication
(10) Pub. No.: US 2007/0066897 A1
(43) Pub. Date: Mar. 22, 2007

(54) Systems and methods for performing acoustic hemostasis of deep bleeding trauma in limbs

(76) Inventors: K. Michael Sekins, Yarrow Pt., WA (US); John Kook, Seattle, WA (US); Ed Caldwell, Everette, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/486,526

(22) Filed: Jul. 13, 2006

Related U.S. Application Data
(60) Provisional application No. 60/699,275, filed on Jul. 13, 2005.
(60) Provisional application No. 60/699,253, filed on Jul. 13, 2005.
(60) Provisional application No. 60/699,290, filed on Jul. 13, 2005.

(51) Int. Cl. A61B 8/00

(52) U.S. Cl. 600/437

(57) ABSTRACT

An inflatable cuff having integrated ultrasound transducers is used to effect hemostasis of deep bleeding wounds in limbs. The cuff includes a chamber defined by a bladder or a series of dams into which a fluid may be introduced and pressurized. The pressure of the fluid stops or slows bleeding while high intensity focused ultrasound is applied to effect hemostasis. The fluid may also serve as an acoustic couplant between the limb and the ultrasound transducers. The transducers may be electrostrictive transducers. Diodes may be used to reduce parallel capacitive loading in the transducer array. Bypass capacitors using the electrostrictive material may also be used.

### CROSS-REFERENCE TO RELATED APPLICATIONS

The present application claims priority to U.S. Provisional Application 60/699,275 filed on Jul. 13, 2005, 60/699,253 filed on Jul. 13, 2005 and 60/699,290 filed on Jul. 13, 2005, all of which are incorporated herein by reference in their entirety.

### STATEMENT REGARDING FEDERALLY SPONSORED R&D

The U.S. Government has a paid-up license in this invention and the right in limited circumstances to require the patent owner to license others on reasonable terms as provided for by the terms of Contract No. W81XwH-06-C-0061 entitled “Noninvasive Acoustic Coagulation System for Life Threatening Battlefield Extremity Wounds” awarded by the Defense Advance Research Projects Agency (DARPA).

### BACKGROUND OF THE INVENTION

1. Field of the Invention

The present disclosure is directed generally to systems and methods for performing acoustic hemostasis on bleeding trauma in limbs.

2. Description of the Related Art

Certain injurious events result in bleeding penetration wounds in the limbs of the human body, for example military combat bullet and shrapnel wounds, vehicular accidents, and insertion of penetrating devices (needles and catheters) into tissue during medical procedures, such as blood vessels and/or organs. Following such injuries, it is desirable to rapidly stop the bleeding from these wounds (hemostasis), especially bleeding from puncture wounds of significant blood vessels, and to do so in an efficient manner, minimizing time and effort.

### SUMMARY OF THE INVENTION

One embodiment disclosed herein includes an ultrasound cuff comprising an array of ultrasound transducers adapted to be deployed circumferentially around a body limb and a pressurizeable bladder positioned on a first side of the array, wherein the cuff is configured such that when the array is deployed circumferentially around the body limb, the bladder is positioned between the array and the body limb.

Another embodiment disclosed herein includes an ultrasound cuff that includes a flexible array of ultrasound transducers adapted to be deployed circumferentially around a body limb and at least one seal positioned along at least one edge of at least some of the ultrasound transducers, the seal configured to contact the body limb when the array is deployed circumferentially around the body limb such that a space is maintained between the body limb and the at least some transducers.

Another embodiment disclosed herein includes a method of effecting hemostasis in a wound in a limb comprising placing an inflatable cuff with integrated ultrasound transducers around the limb, inflating the cuff, and applying high intensity focused ultrasound sufficient to effect hemostasis with the transducers.

Another embodiment disclosed herein includes an method of effecting hemostasis in a wound in a limb comprising placing a tourniquet on the limb, placing a cuff with integrated ultrasound transducers around the limb, and applying high intensity focused ultrasound sufficient to effect hemostasis with the transducers.

Another embodiment disclosed herein includes an ultrasound applicator patch comprising an array of ultrasound transducers, sidewalls disposed around the perimeter of the array, wherein the sidewalls define a space over the ultrasound transducers, and an inlet port in the sidewalls or the array configured to allow introduction of a fluid into the space.

Another embodiment disclosed herein includes a method of effecting hemostasis in a wound comprising positioning the patch described above over the wound, introducing a fluid through the inlet port into the space, and applying high intensity focussed ultrasound sufficient to effect hemostasis with the transducers.

Another embodiment disclosed herein includes an ultrasound applicator patch that includes an array of ultrasound transducers and a pressurizeable bladder positioned on a first side of the array, wherein the cuff is configured such that when the array is deployed on the wound, the bladder is positioned between the array and the wound.

Another embodiment disclosed herein includes a method of effecting hemostasis in a wound that includes positioning the patch described above over the wound, introducing a fluid into the pressurizeable bladder, and applying high intensity focused ultrasound sufficient to effect hemostasis with the transducers.

### BRIEF DESCRIPTION OF THE DRAWINGS

FIG. 1 schematically illustrates a cross section view of a deep bleeder acoustic coagulation (DBAC) cuff system that is minimally pressurized in order to accomplish acoustic coupling.

FIG. 2 schematically illustrates the cuff system of FIG. 1 in an increased pressurized state in order to either partially or totally occlude blood vessels, thereby limiting perfusion of the limb and allowing for efficient application of HIFU for therapeutic acoustic hemostasis.

FIG. 3 schematically illustrates a single closed bladder cylindrical wrap cuff configuration for a DBAC cuff system.

FIG. 4 schematically illustrates a proximal-distal dam with a coupling chamber cuff configuration for a DBAC cuff system.

FIG. 5 is a perspective view of a proximal-distal dam with a coupling chamber cuff configuration for a DBAC cuff system.

FIG. 6 is a perspective view of the cuff system of FIG. 5 illustrating one architecture that allows accommodation of different limb sizes.

FIG. 7 is a perspective view of another deep bleeder acoustic coagulation cuff embodiment using individual water bladder for each array panel.

FIG. 8 is a perspective view of another deep bleeder acoustic coagulation cuff embodiment using individual water dams for each array panel.

FIG. 9 is a perspective view of a limb having a deep bleeding penetration wound and a Deep Bleeder Acoustic Coagulation patch.

FIG. 10 is a perspective view of the interior side of the Deep Bleeder Acoustic Coagulation patch of FIG. 9.

FIG. 11 is a schematic of an electrostrictive transducer array architecture.

FIG. 12 is a perspective view of a crossbar interconnect in a 2D array of transducer elements.

FIGS. 13A and 13B are schematics illustrating one channel detection and driving circuits using two different DC bias connection methods.

FIG. 14 is an electrical schematic of one sub row of a 2D array of transducer elements.

FIG. 15 is an electrical schematic illustrating two different diode interconnect schemes.

FIG. 16 is an electrical schematic illustrating one sub row of a 2D array of transducer elements with an extra +10 volt bias line.

FIG. 17 is a perspective view of a crossbar interconnect in a 2D array of transducer elements with extra current source bias lines.

FIG. 18 is a perspective view of a 2D array of transducer elements with connected diodes.

FIG. 19 is a perspective view of a 2D array of transducer elements with connected diodes including a conductive kerf fill.

FIG. 20 is a perspective view of another 2D array of transducer elements with connected diodes including a conductive kerf fill.

FIG. 21 is a perspective view of another 2D array of transducer elements with connected diodes including a low voltage bias strip.

FIG. 22 is a top view of the 2D array of FIG. 21.

FIG. 23 is a cross section of the 2D array of FIG. 21.

FIG. 24 is a schematic illustrating typical electrostrictive transducer element interconnection having a bias supply and driving signal interconnects.

FIG. 25 is a schematic illustrating an interconnection scheme for an array of electrostrictive elements.

FIG. 26 is a perspective view of a 20×20 electrostrictive array having bias supply and driving signal interconnects.

FIG. 27 is a perspective view of an electrostrictive capacitor assembly.

FIG. 28 is a perspective view of an electrostrictive capacitor array located on top of an electrostrictive transducer array.

FIG. 29 is a schematic illustrating a typical electric circuit in an electrostrictive transducer array.

FIG. 30 is a schematic illustrating electrostrictive transducer elements on a high voltage strip.

FIG. 31 is a circuit diagram illustrating electrostrictive transducer elements on one high voltage rail strip.

FIG. 32 is another circuit diagram illustrating electrostrictive transducer elements on one high voltage rail strip.

### DETAILED DESCRIPTION OF CERTAIN EMBODIMENTS

Inflatable Cuff with Ultrasound Applicator for Hemostasis

Various embodiments provide devices and methods for optimally treating bleeding wounds in injured limbs with acoustic hemostasis using high intensity focused ultrasound. It has been discovered that raising tissue and blood temperatures to threshold levels for a sustained and appropriate dosing time can be used to stop traumatic bleeding in a controlled, reliable manner. Although blood coagulation can be achieved at modest sustained elevated temperatures (≈T>46° C.), at these temperatures, a long heating time is required and blood itself is a poor absorber of ultrasound energy (e.g., α_{blood}≈0.03 Np/cm/MHz as compared to α_{muscle}≈0.15 Np/cm/MHz where α is the acoustic absorption coefficient. Therefore, capillary and vessel sealing are largely linked to collagen denaturation and shrinkage associated with higher sustained temperatures (≈T>70° C.). Tissue temperatures much higher than these can lead to non-linear behaviors (e.g., cavitation, boiling, enhanced attenuation, reflection etc.), rendering safety and control of the hemostasis process more difficult. It is extremely difficult to achieve hemostasis if dosing (acoustic energy delivery) occurs in the presence of significant intravascular blood flow (blood flowing through vessels) and/or extravascular blood flow (bleeding). Such flow dramatically dissipates thermal energy deposition, producing either subtherapeutic temperatures or a requirement to compensate with large increases in acoustic power and dosing time. Such elevated power places the skin and “interpath” tissues at higher risk for thermal/acoustic injury such as burns, obliterative coagulative necrosis, and tissue cavitation. In addition, control is rendered more challenging in light of the non-linear effects mentioned. Finally, such elevated power creates greater challenges on device thermal management, thermal stress, power-associated system weight and other related factors.

To address these complications, it is desirable to minimize bleeding from the target injury site(s) during dosing. In the case of extreme combat trauma (e.g. fast bleeders in multiple vessels), it may be advantageous to dose in the absence of limb perfusion altogether. By minimizing the injury bleeding and perfusion one can deposit the acoustic energy at the bleed site without having to compensate for energy being swept away through bleeding in the treatment volume. Such bleeding dissipates the acoustic energy, which lowers the tissue temperatures during the power dose period. The effect of this dissipation is a) to reduce efficacy (e.g. reduced coagulation and collagen cross-linking, mechanisms that help with affecting the seal) due to lower temperatures, or b) to require the addition of significant therapeutic acoustic power to maintain the desired therapeutic temperatures.

Accordingly, in one embodiment, a liquid and/or gas inflatable compartment (bladder) is integrated into a deep bleeder acoustic coagulation (DBAC) acoustic hemostasis cuff device. The inflatable subsystem and its control module may be used to apply compression to slow or stop bleeding during application of the acoustic therapy. This system enables the deposition of the acoustic energy at the bleed site without having to significantly compensate for energy being swept away through bleeding. Surface power reduction also lowers the risk of the patient/subject (e.g., injured soldier) experiencing burning of the skin and “interpath” tissues. The cuff may be used to provide an on-demand tourniquet so that bleeding may be minimized during device setup, treatment delays, or interruptions, thereby buying additional time in regard to risk of shock period. The cuff also permits delivery of pressure preferentially to the treated limb. A control module may be configured to automate cuff inflation and control. Additionally, the cuff may also serve as an on-demand splint for limb immobilization.

An additional benefit of providing compression during therapy is that the coupling of the sound to the treated limb for therapeutic energy delivery and acoustic targeting and detection is enhanced. Furthermore, the pliant nature of some embodiments of the cuff device facilitates acoustic coupling to tissues with irregular shaped surfaces (either normal skin, or open wounds) or limbs of various sizes. In other embodiments, the cuff device facilitates acoustic coupling by conforming to the body via liquid instead of a pliant mechanical cuff as described in more detail below.

In some embodiments where the cuff is inflated with a liquid, flow of the liquid through the cuff may be used to provide surface cooling of the skin or the wound surface of the tissue being treated, thereby further mitigating potential superficial burns. Fluid flow through the cuff may also be used to cool the array transducer surface thereby improving performance and device reliability. In addition, the cuff, whether liquid or gas filled, can be used to provide a thermal stand-off to separate a potentially hot therapeutic applicator surface from the skin.

In one embodiment, the cuff architecture may be used to provide an outer structural layer (e.g., an exoskeleton), thereby giving a fixed and (relative to the limb treatment volume) immobile support for the detection and therapy transducer arrays. In addition, the architecture may be used to control and fix the limb shape for optimal acoustic therapy as well as to provide an on-demand splint.

In one embodiment a cuff system is provided that includes components which control limb and injury bleeding during dosing and/or during detection/localization. In one embodiment, the cuff system includes a liquid-inflated compartment (bladder) surrounding the limb to be treated, with an accompanying pressurization control system. Because the liquid compartment can deliver pressure to the limb, it can both constrict limb blood flow during dosing (like a conventional blood pressure cuff at peak pressure) as well as permit controlled limb perfusion during bleeder detection when cuff pressure is released.

In automated modes the cuff pressurization can be stepped through inflation-deflation cycles in a programmed manner, and can hold pressures at desired levels (e.g. just below systole to allow big bleeders to be detected with less blood loss). Low frequency flow-disturbance associated noise in vessels, such as the well-known Korotkoff sounds used in blood pressure cuff pressure-release maneuvers, can be used as potential indicators of appropriate applied pressure for hemostasis of major arteries. Other low frequency sounds may also be useful in automated pressure control of the DBAC cuff, such as used in “vibrometry” detection of tissue and vessel wall motions. By controlling cuff pressure delivery and release venting rates, and using acoustic motion tracking algorithms, the motion of the bleeding targets in the transition between high and low cuff pressure states can be monitored. Very little cuff-tissue motion of bleeders need occur between detection and treatment phases. Further, Doppler signals from bleeders as a function of applied cuff pressure will have diagnostic value in prioritizing bleeder targets.

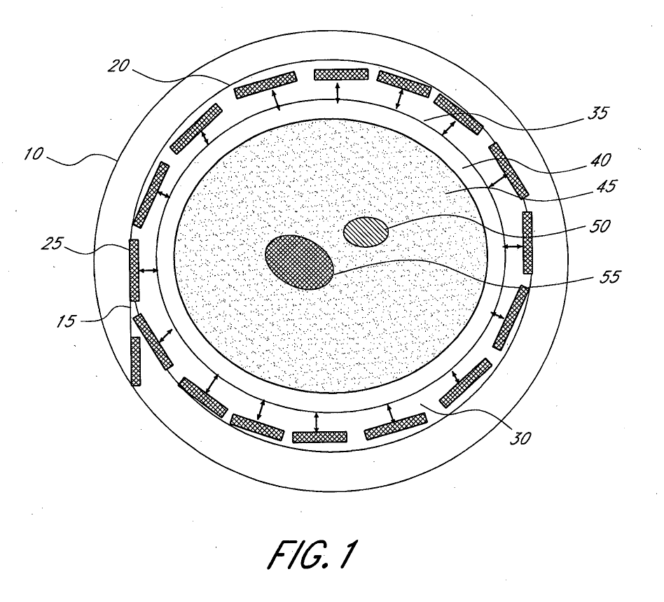

FIG. 1 illustrates a cross-sectional view of one embodiment of a DBAC cuff system having a liquid-inflated coupling and pressure delivery compartment/chamber illustrated in a low pressure state. An outer cuff material 10 is provided on the exterior of the cuff. The outer cuff material 10 may include a circumferential lock system (e.g. velcro, zipper, snaps, tie, etc.) to provide a restriction on the maximum outer diameter that the cuff can obtain. A cuff and transducer array overlap area 15 allows the cuff to fit various limb circumferences. Transducer elements in the overlap area 15 may be non-activated during use. A semi-rigid sheath 20 may be provided that serves to constrain the transducer array elements 25 and may also provide intra-array linkages. This semi-rigid sheath 20 may be composed of a stiff durable polymer (e.g. HDPE (high density polyethylene)) that serves as a firm foundation (exoskeleton) for directing pressure to the treated limb. Transducer elements 25 may be an individual imaging and/or therapeutic transducer array modules capable of acoustic detection, localization, targeting and/or therapeutic hemostasis. Methods of using ultrasound transducers for detection, localization, targeting, and therapy are known in the art and any suitable method may be used with the DBAC systems described herein.

A liquid inflatable compartment 30 (e.g. cylindrical bladder filled with degassed water) provides adjustable compression to the limb. In the case that the compartment 30 is liquid filled, it may be acoustically coupled to the body, thereby transmitting acoustical energy from the transducers 25 to the limb. A configuration which utilizes a proximal and/or distal dam configuration (described below) may utilize either gas or liquid to inflate the pressurized dams. In both cases fluid may serve to acoustically couple ultrasound between the transducer elements 25 and the body as well as apply pressure to the limb. The patient's limb includes skin surface 35, subcutaneous fat layer 40, and muscle 45. Artery 50 and bone 55 are within the limb.

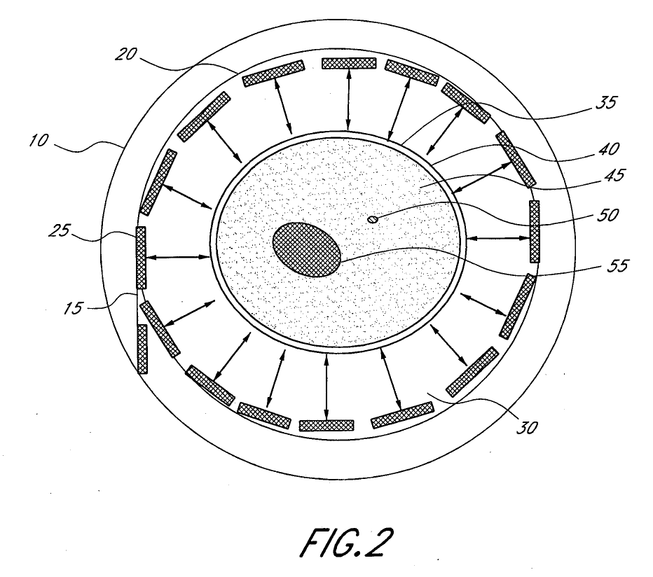

FIG. 2 illustrates the same cross-sectional view as FIG. 1, however, with the compartment 30 pressurized. This pressurization either occludes or partially occludes the artery 50 in order to minimize bleeding and blood flow during the power delivery (dosing) period (e.g., to effect acoustic hemostasis). As depicted in FIG. 2, the diameter of the artery 50 has been reduced due to the inflation pressures of the cuff. When the compartment 30 is pressurized, the transducer array 25 does not move significantly relative to its unpressurized position due to the semi-rigid sheath 20, which minimizes the movement of the array 25 and outer cuff material 10 outward away from the limb due to the pressurization.

The inflatable compartment 30 holds (and delivers) pressure forces. The pressure can be delivered by providing features in the cuff that enable on-demand or automated, cuff inflation that constricts limb flood flow, in a tourniquet like fashion, e.g. similar to the operation of conventional blood-pressure cuffs that are used with Korotkoff sound detection of the sequential shutting off of blood flow and its resumption. The inflatable compartment 30 serves as an on-demand (i.e., quickly deployed and reversed) tourniquet, playing a key role in preserving patient/soldier blood volume during treatment preparation, treatment bleed site detection, or treatment delays or interruptions.

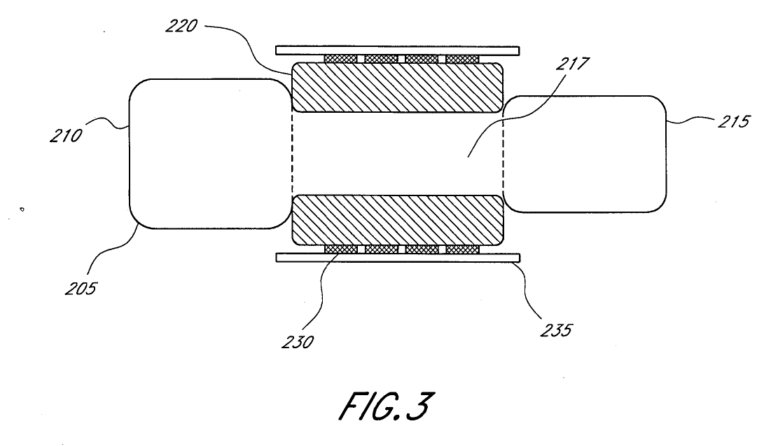

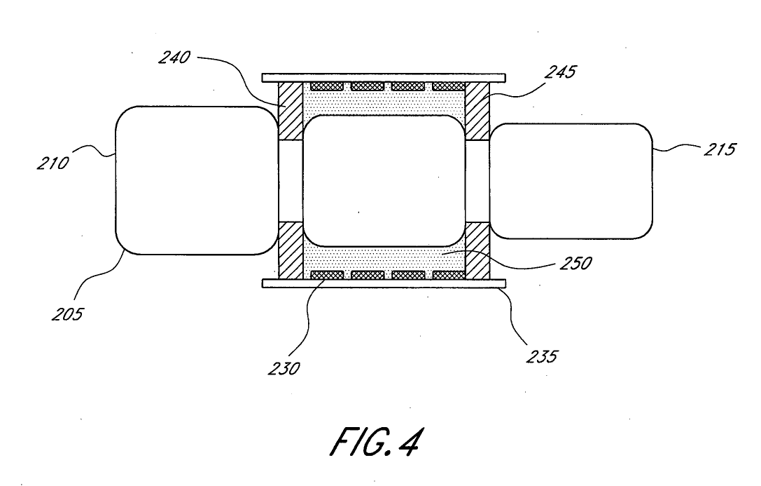

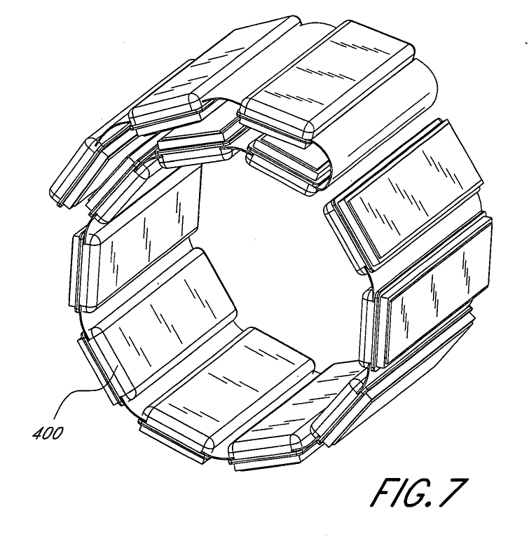

The inflated liquid-chamber architecture of the cuff device can be implemented in a variety of configurations. FIGS. 3 and 4 shows two optional configurations (shown in the pressurized state). A single closed bladder cylindrical wrap cuff configuration is illustrated in FIG. 3. This cuff configuration is deployed on a patient limb 205 having a proximal side 210 and a distal side 215. The bladder 220 is configured as a single closed liquid-filled chamber that surrounds the limb segment to be treated 217. Alternatively, the bladder may be composed of multiple separate bladders as discussed below and illustrated in FIG. 7. The liquid chamber 220 serves to deliver the desired pressure, couple the acoustic energy to the tissue, insulate the patient's skin from potentially hot transducer surfaces, and optionally cool or sink heat away from the skin during acoustic dosing. In some embodiments, an acoustic couplant is positioned at the interface between the chamber membrane and the patient skin (e.g., a gel or a gel pad) in the treatment area of the limb. As illustrated, the exoskeleton sheet 235 is connected to the ultrasonic transducers 230. An acoustic couplant (e.g., a gel or gel pad) can be used to couple the acoustic energy from the transducers 230 to the bladder 220. An alternative configuration is to enclose the transducers 230 within the bladder 220, thereby eliminating the need to use an acoustic coupling media between the transducer and the bladder.

FIG. 4 illustrates a configuration utilizing a proximal-distal dam with a coupling chamber cuff. The inflatable chamber is segmented into 3 sections, a) the proximal dam 240 at the proximal limb side 210, b) the distal dam 245 at the distal limb side 215, and c) the central coupling chamber 250 filled with liquid. The ultrasound transducers 230 attached to the exoskeleton sheet 235 are in direct contact with the liquid in the central coupling chamber 250 and thus are directly acoustically coupled to the limb 205. In this configuration, the proximal and distal dams 240 and 245 are controlled-pressure sections of the cuff enabling the central section to remain sealed and liquid-filled on the limb 205 by maintaining a pressure seal against the skin. The proximal and distal dams 240 and 245 may include ring or torus shaped bladders that can be inflated to a pressure P_{dam} to maintain the pressure seal. Because in one embodiment there are no transducers located directly behind the dams there is no need for acoustic coupling is needed through the dams 240 and 245, and thus gas may be used as in the inflation medium within the dam bladders. However, in an alternative embodiment it may be advantageous to have ultrasound transducers behind the dam, in which case such dams may be pressurized with acoustic coupling liquid (e.g. water).

In the configuration of FIG. 4, no separate acoustic couplant medium is required at the skin interface. The central coupling chamber 250 where the acoustic treatment and targeting paths occur may be made substantially free of air, thereby enabling good acoustic coupling. During acoustic dosing, the liquid in the central chamber 250 can be kept at a pressure, P_{coupling}, which can be high enough to slow or stop blood flow as described above. However, the pressure is advantageously low enough to maintain the cuff proximal and distal seals (e.g., P_{coupling}<P_{dam}). It is also noted that tourniquet action can be delivered by either the proximal or distal dams 240 and 245 and that, further, the dam segments can be inflated by either a gas (e.g., air) or by a liquid. In addition, in some alternative embodiments, more than two inflatable seals may be provided.

As discussed above, the liquid used to inflate the chambers positioned beneath the ultrasound transducers serves both the function of providing pressure to shut off (or restrict) the injury blood flow in the limbs as well as to enhance coupling of sound from the transducers to the treated limb for therapeutic energy delivery and acoustic targeting and detection. The coupling fluid may be any fluid having suitable acoustic transmission properties. In some embodiments, the fluid is water or physiologic saline (e.g., sterile, or non sterile, degassed or non-degassed water). In embodiments utilizing dams such that the coupling liquid is in direct contact with the limb/wound, pro-coagulant and/or anti-infection agents may be included in the coupling liquid to further promote hemostasis while reducing the risk of infection. A non-limiting example of a suitable pro-coagulant is thrombin. A non-limiting example of a suitable anti-infective is an antibiotic.

In addition to providing an acoustic path to the tissue, the liquid-filled compartments of the cuff also facilitate coupling in a compliant manner to tissues with irregular shaped surfaces (either normal skin, or open wounds) or limbs that vary in size (e.g., diameter). That is, the liquid-filled fluid bolus that comprises the inflated portion of the cuff would be able to accommodate the contours of the limb surface while performing acoustic coupling and limb compression force delivery.

Additionally, the cuff fluid compartment also provides, through forced convection or natural convection, surface cooling to the skin or the wound surface of the tissue being treated, thereby mitigating potential superficial burns due to the therapeutic ultrasound dosing. In some embodiments, these functions can be enhanced by providing a temperature-controlled, recirculating supply of liquid to the cuff. In some embodiments, the liquid is further processed, such as by providing degassing mechanisms to enhance the acoustic coupling properties of the liquid. The fluid compartment also serves as a thermal stand-off to separate hot therapeutic ultrasound applicator surfaces from the skin, thereby minimizing conductive heating (in addition to ultrasound absorption) contributions to superficial burn risk. Further, the fluid convection also controls the temperature of the applicator surface, potentially optimizing acoustic transducer performance and reducing thermal failure or device lifetime risks.

Some embodiments include a control system that allows cuff pressurization (or, equivalently, inflation volume) to be varied (either manually or automatically) according to whether, a) blood limb perfusion should be prohibited (or reduced), as needed for dosing requirements, b) the pressure/volume in the cuff should be reduced to permit appropriate blood flow in vessel lumens for bleeding detection and therapeutic targeting, or c) bleeding from the injury site needs to be controlled (e.g., pressurization only permitting peak systolic pressure event bleeding). Such a variable control (manual or automatic) of the cuff further enhances detection, targeting/localization, and coagulation treatment.

To enable effective application of cuff pressure for coupling, skin and transducer cooling, and bleeding control, control systems may be provided that both allow the user/operator to manually moderate the cuff inflation and that step through cuff inflation pressures in a programmed manner, alternatively localizing bleeds (during detection and localization/targeting phases) using lower inflation level periods, and then being set to higher inflation levels during dosing.

In one embodiment, the pressure delivery aspects of the inflatable cuffs may be used for controlling and fixing the limb shape for optimal acoustic therapy. For example, the cuffs may be used to put bleeder targets within optimal depth ranges for the multiple transducer modules in the cuff. In some embodiments, cylindrical or oval cross-sectional shapes for the limb may be optionally imposed via cuff pressurization strategies.

In one embodiment, the array of acoustic transducer modules is coupled to the liquid-filled compartment by having the transducer aperture surfaces protrude through the external membrane wall of the liquid-inflated chamber (FIG. 3). In this manner, no additional couplant (e.g., gel) is needed at the transducer-coupling chamber surface. As discussed above, matrix transducer elements (including their acoustic backing layers) may be mounted on a thin bendable sheet of stiff material (e.g., a polymeric material) that effectively constrains the array matrix and serves as a foundation (i.e., an exoskeleton) reacting against the inflation pressure in the cuff so that pressure is preferentially delivered to the treated limb. This bendable sheet exoskeleton is able to wrap around the contours of the limb, backing up the array. In some embodiments, the exoskeleton is surrounded by a cloth material layer, which can be fixed in place with Velcro-type strapping or other fixture mechanisms to hold the cuff in place on the limb. Thus, the inflation of the cuff can be used during dosing to give a fixed and (relative to the limb treatment volume) immobile architecture to the detection and therapy transducer arrays.

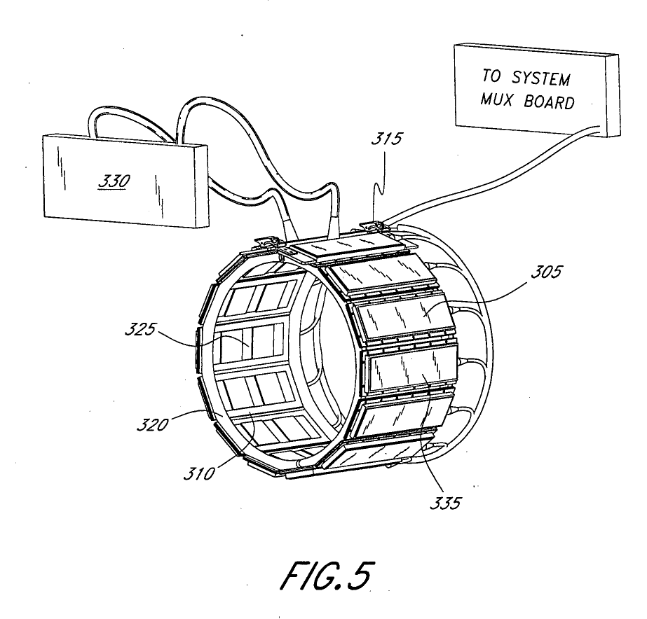

FIG. 5 illustrates one embodiment of an acoustic hemostasis inflatable cuff. The cuff in FIG. 5 has a 25 cm diameter×40 cm cylindrical length. The cuff is composed of ultrasonic transmitter/receiver array panels 305. Each panel 305 contains several array tiles which are made up of individual piezoelectric acoustic elements. These tiles can be sized and oriented to maximize acoustic penetration, coverage, detection and localization of the treatment area. The panels can be made to be mechanically independent and connected via a flexible membrane/hinge 310 to facilitate wrapping around the limb. A locking mechanism 315 is provided on the cuff in order to close the cuff around the limb. The cuff may be sealed onto the limb using proximal/distal dam air bladders 320 that form a complete chamber within the cuff. This air bladder/water dam 320 is located around the circumference of both ends of the cylindrical cuff. The volume that the bladders define is flooded with water which serves as the coupling fluid and heat sink for the array 325 face and patient skin. Those of skill in the art will appreciate other acoustic coupling fluids that may be used in volume. The array panels 325 are designed to be water proof so as not to be affected by water that leaks around the air bladder seal or is splashed during installation and disassembly. Cooling of the coupling water may be accomplished via the use of a liquid (e.g. water) recirculating system 330. The system 330 may also be used to introduce water into the volume, to pressurize the water, and to degas the water. Additional cooling may be accomplished via the use of electronic devices (e.g. Peltier devices 335) mounted on the outside surface of the transducer panels. Other methods of cooling are possible such as convection cooling over heat sink fins.

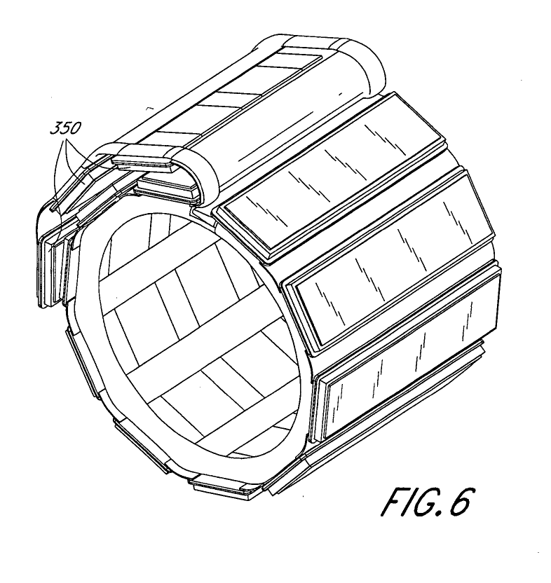

FIG. 6 illustrates one embodiment that accommodates different sizes of limbs. The transducer panels are connected to each other via a stretchable membrane. As in FIG. 5 a water dam provides for a flooded chamber between the transducer element and the skin. The water dam/air bladder is pinched off and sealed through the process of folding the unused panels 350 back on the panels in use. The water dam is not inflated until the cuff is in place and the air bladder is pinched off.

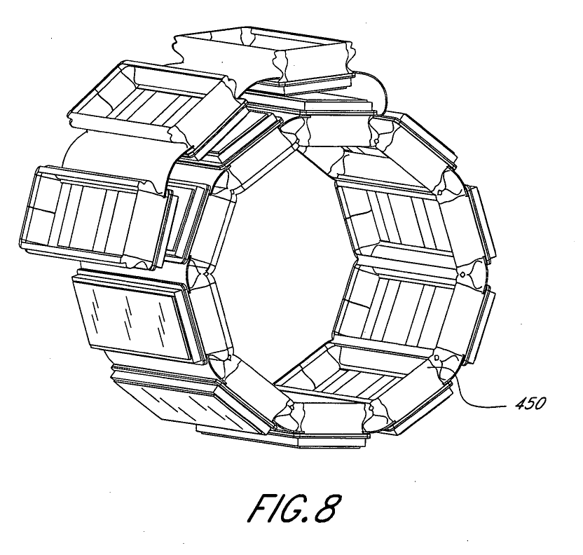

In FIGS. 5 and 6, a water volume is contained within a continuous compartment. Alternative embodiments are illustrated in FIGS. 7 and 8. In FIG. 7 the water barrier and acoustic coupling is accomplished using an individual water bladder 400 for each array panel. This configuration forms a fluid coupling chamber independent of adjacent panels. In some embodiments, a hydrogel or other solid acoustic couplant could be used in place of each individual water bladder. In FIG. 8 seals 450 are utilized for each array panel to create a dam in order to contain water or other acoustic couplant. These seals may be made of pliable materials such as silicone or foam (e.g., such as the seals used on swim goggles or underwater diving masks). In one embodiment, a skin compatible quick set foam is used to form a water dam. Such a quick set foam may be similar to the sprayed in place urethane foams that are used as thermal and acoustic insulation. The foam may be optimized to provide fast set time and the appropriate softness. Another embodiment utilizes an expandable sponge or hydrowicking material that expands to form a watertight barrier once it contacts water.

Using independent water panels as described above provides the advantage of being able to remove a panel and still have the cuff function (i.e., independence). In addition, if one panel's dam or individual water bladder breaks or is damaged, neighboring panels may be sufficient to provide acoustic hemostasis, acoustic bleeding detection, and the ability to reduce and/or stop bleeding via applied pressure (i.e., redundancy is built into the system). Finally, the flexible material connecting the panels to each other does not have to be water tight allowing a wider range of materials to be used between panels in order to provide for flexibility.

In some embodiments, the cuffs described above may be used by first placing an injured patient/soldier in an appropriate treatment position. A disposable sterile barrier pad may be then be wrapped around the injured limb. The barrier pad may include acoustic coupling properties such as acoustic gel prepositions on both sides of the barrier. A deep bleeder acoustic coagulation (DBAC) cuff is then unrolled and wrapped onto the injured limb over the disposable pad and locked or strapped snugly into position. Electrical and fluid connections from the cuff to the base unit (RF power, control system, and fluids subsystem) may then be made. Alternatively, the connections may be pre-connected to save procedure time. Similarly, the fluid compartments and fluid lines may be pre-primed where possible.

Fluid compartment(s) in the cuff may be first pressurized with manual activation to achieve (a) good acoustic contact between the cuff, disposables, and limb, (b) a stable and semi-rigid deployed configuration of the cuff system and limb, and (c) hemorrhage control through cuff pressurized tourniquet action. The cuff fluid will occupy all of space between limb and conformal transducer array blanket layer. After manual activation, the operator can initiate automatic treatment, which may include repeated cycles of detection, localization, and HIFU therapy until all bleeders are sealed. The automatic programming may also adjust the pressure of the cuff in order to achieve the desired functions of detection, localization, and therapy.

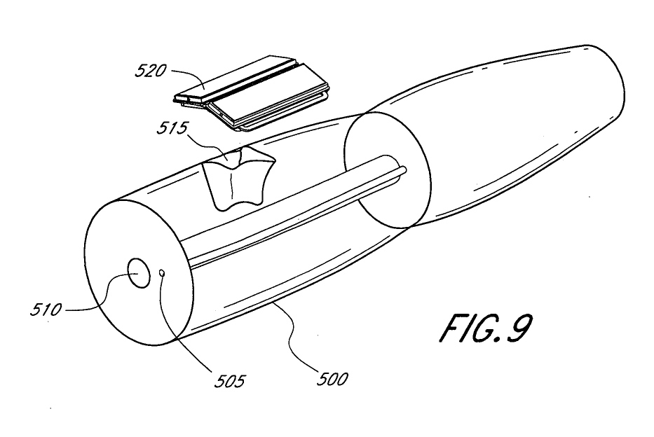

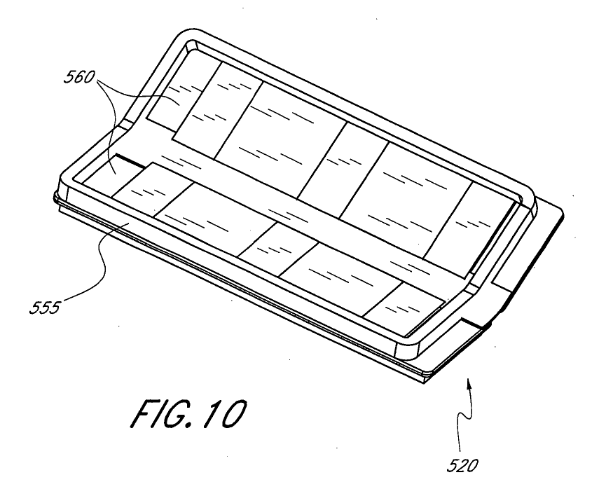

FIGS. 9 and 10 illustrate another embodiment where only a portion of a cuff or a “patch” 520 is applied to the injured area 515 of the limb 500 or other body part (e.g. torso, neck, etc.) and is capable of acoustic detection, localization, targeting and therapeutic hemostasis via high intensity focused ultrasound. In one embodiment, the patch design provides a seal with the skin by using a very aggressive adhesive 555 so that the patch does not require any additional mechanical means to keep it in place. The area 560 under the patch can then be flooded for acoustic coupling to injured vessels within the cavity. An advantage to the deep bleeder acoustic coagulation patch is its light weight and portability. Use of such a patch is not limited to limb trauma and may be applied to other portions of a patient (e.g., the torso).

Ultrasonic Array and System for Acoustic Hemostasis

The ultrasound transducers for use with any of the above described arrays may include but are not limited to conventional PZT ceramic transducers, electrostrictive transducers, capacitive microfabricated ultrasonic transducers (cMUTs), and PZT microfabricated ultrasonic transducers (pMUTs). The above systems may utilize a single set of transducers that perform low power ultrasonic detection/localization as well as high power High Intensity Focused Ultrasound (HIFU) functions. Alternatively, two sets of ultrasonic transducers may be provided for the separate purposes of optimized detection/localization and therapy. These two sets of transducers may be made of the same piezoelectric material (e.g. PZT, electrostrictor, cMUT or PMUT) or they may be a hybrid combination (e.g. a hybrid architecture whereby cMUT 2-D imaging arrays are used for detection/localization and are interlaced with the electrostrictive transducers for therapy).

In conventional 2D array designs based on PZT ceramics, the interconnection complexity is a challenge due to the small size and the large numbers of the elements. A typical 2D array with λ/2 spacing has over 4000 electrical connections, if fully sampled. This problem is exacerbated in the DBAC cuff, which is 40 cm by 80 cm in dimension. For example, a cuff operating at 1 MHz for therapy and imaging with a 0.8 mm pitch has potentially 500,000 electrical connections. These element numbers also significantly increase the multiplexing circuit complexity, implying numbers of active channels that are impractical. The DBAC driving circuit requirements add further challenges since the optimal circuit design for bleeder detection and that for therapy delivery may be different. Accordingly, in some embodiments, architectures are provided that allow for the simplification of the system complexity via the use of transducer choice and overall cuff design. Non-limiting examples of architectures that may be used include:

Electrostrictive Array Architecture: This approach uses electrostrictive transducers exclusively, with each transducer used alternatively for detection/localization and therapy. The detection and localization approach may use Doppler interrogation of the limb. The bias controlled architecture enabled by electrostrictive materials produces significant simplifications viz. PZT piezoceramic devices when it comes to channel count and interconnect complexity.

cMUT Array Architecture: This approach uses cMUTs for detection/localization and therapy, providing both therapeutic power and 3D-based targeting. This approach is architecturally similar to the electrostrictive array approach with bias control used to reduce channel count and interconnect complexity.

pMUT Array Architecture: This approach uses pMUTs for detection/localization and therapy, providing both therapeutic power and 3D-based targeting via pMUTs. This approach is architecturally similar to the electrostrictive and cMUT approach with bias control used to reduce channel count and interconnect complexity.

PZT Array Architecture: This approach uses PZT for detection/localization and therapy. This approach is potentially challenging given the high channel/interconnect count, however, micro-mechanical switches can be used to provide for a simplified design.

Hybrid Architecture: This approach uses a hybrid architecture whereby either cMUT, pMUT, PZT or Electrostrictive 2-D arrays are used for detection/localization, and are interlaced with a different type of transducer for therapy.

Unlike normal piezoelectric materials (e.g., PZT), electrostrictive materials (also termed “relaxors”) require a DC bias voltage to exhibit piezoelectric properties. When the DC bias voltage is removed, the field-induced polarization disappears and the material ceases to be piezoelectric. This means that entire groups of transducers can be turned on or off by application or removal of the bias field. As described below, this enables the number of driver channels to be greatly reduced, simplifying interconnection and control issues significantly, as well manufacturing cost and complexity. While the potential of electrostrictive materials have been demonstrated in both medical and sonar applications, commercial development has been slow due to problems encountered when attempting to implement electrostrictive transducers.

In recent years, the field-induced piezoelectric property of electrostrictive materials has been explored for medical imaging applications. Several families of relaxor-type electrostrictive materials have been studied for medical imaging applications. Of these, lead-magnesium-niobate modified with lead titanate (PMN-PT) relaxors exhibit the most desirable properties. The advantageous properties of PMN-PT materials for ultrasonic applications include large field-induced piezoelectric coefficients, comparable to PZTs; tunable transmit/receive sensitivity by adjusting the DC bias; high dielectric constant, which improves electrical impedance matching; a spectral response similar to PZT-type transducers; sensitivity and bandwidth comparable to PZT, with slightly higher sensitivity being observed in PMT-PT; relaxor properties conducive to use for both detection and high power therapy; and relatively stable transducer performance over the operating temperature range despite the fact that the dielectric constant and coupling constant is a function of temperature. Three different electrostrictive PMN-PT materials have been developed having operating temperature ranges of 0-30° C., 10-50° C. and 75-96° C., respectively.

Electrostrictive Array Architecture

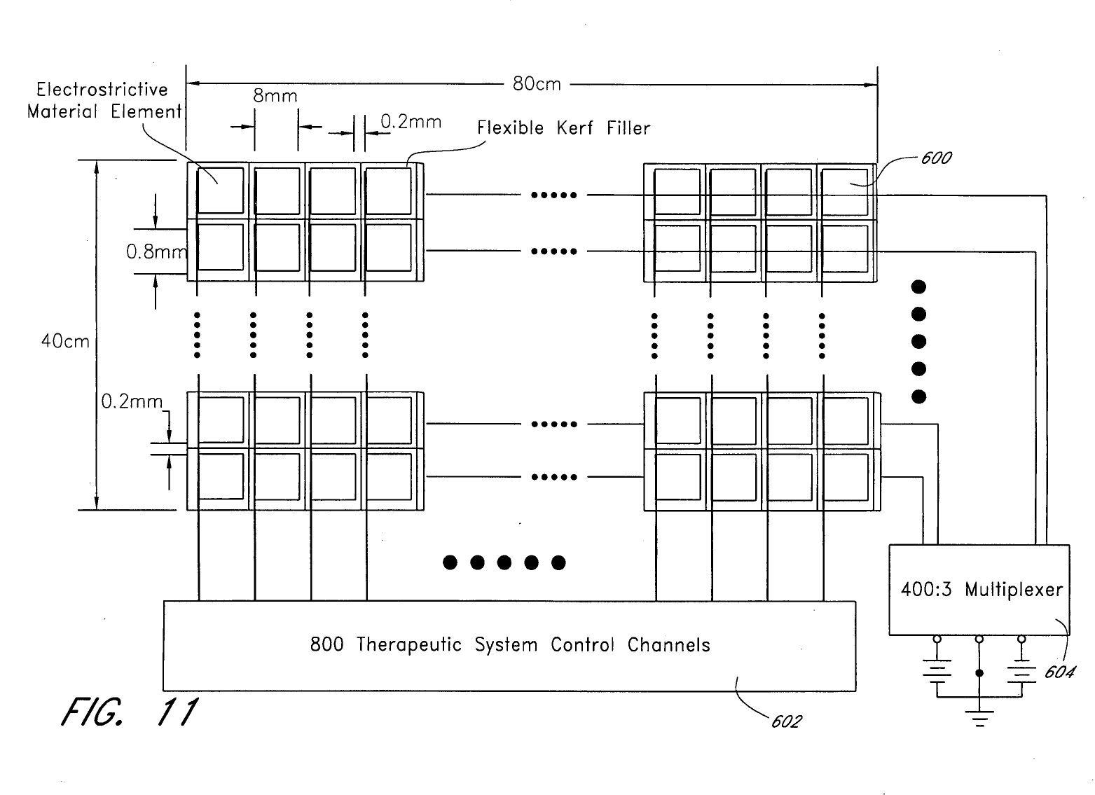

J FIG. 11 is a schematic illustrating the design and bias control of an ultrasonic transducer array based on electrostrictive transducers. This architecture may be used to provide therapy and detection/localization. Operational control of the architecture for detection, localization, and therapy is discussed more fully below. One of skill in the art will appreciate that the electrostrictive array techniques described herein may be utilized in any ultrasound system and are not limited to use in the DBAC cuffs described above.

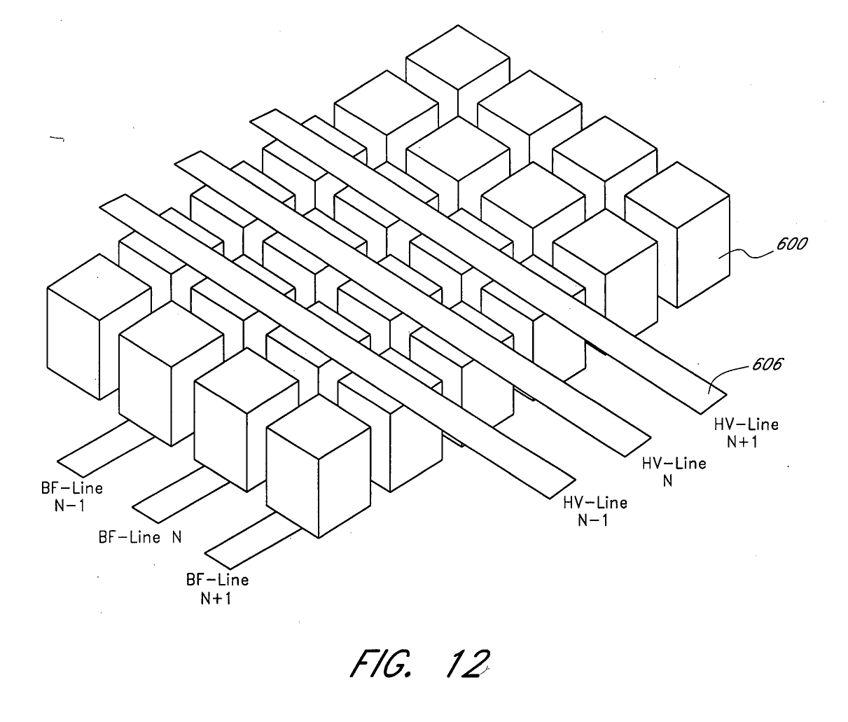

In one embodiment, the architecture shown in FIG. 11 may be used in a 80×40 cm cuff where the relaxor transducer elements 600 cover the entire cuff area. At an operating frequency of 1 MHz, this area would result in an array of 320,000 (800×400) elements. Using the biasing control method to piezoelectrically activate individual rows, only 800 channels are needed to control this array for both Doppler detection/localization and therapy delivery. Along each column, one side (positive) of the elements is electrically connected together to a system control channel 602. Along each row, the back side of the elements 600 are connected to a multiplexer 604. Individual rows of the array are made piezoelectrically active by application of a bias voltage. Control of individual elements 600 along the activated row is via the 800 system channels 602. Furthermore, the polarization direction is varied by using a positive or negative bias voltage. This configuration is also illustrated in a perspective view in FIG. 12, where bias controllable piezoelectric materials (such as electrostrictor or pMUT or cMUT) enable a straightforward crossbar approach to activation of a single element in a 2D array of elements 600.

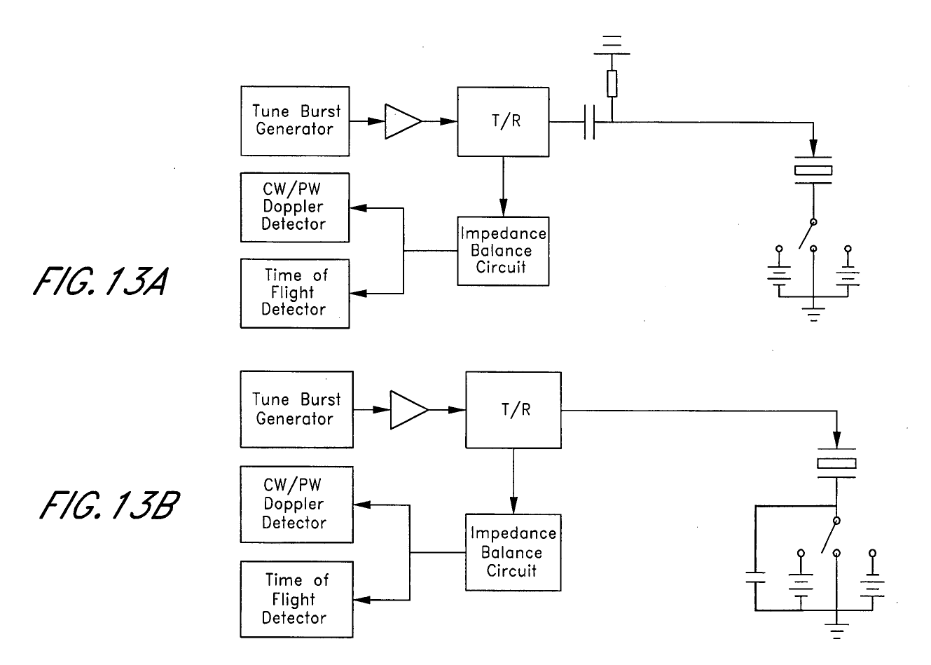

When a row of elements 600 is turned “on”, the focusing position, steering, focal size and power intensity is controlled through the 800 system channel 602. Any elements 600 that have the acoustic path to the bleeder obstructed by bone or metal fragments are turned off through a system channel 602. The selection of the number of rows to be turned on is determined by the depth and size of the bleeder. A Fresnel lens design concept can be used to select the voltage applied to each row. This approach provides the best beam shape for detection, localization and therapy in a cuff architecture. The beam shape and intensity can also be controlled through the magnitude of the DC bias, which shades selective parts of the aperture. For mechanical and acoustic purposes, the relaxor transducers 600 may be grouped in rigidly mounted sub-aperture modules (e.g., 2 cm×2 cm sections) when deployed on a cuff. A detailed function block diagram of one system control channel 602 is shown in FIGS. 13A and 13B.

Use of PIN Diodes for Minimizing Parallel Capacitive Loading

The commercial development of large aperture 2D transducer electrostrictive arrays having thousands of array elements has been limited due to the parallel capacitive loading that the non-activated transducer elements have on the activated elements. This capacitive loading has been identified as a major problem for imaging performance during the imaging receive mode.

In the 2D electrostrictive array described in FIGS. 11 and 12, elements 600 are only activated if a DC high-voltage bias is applied by the horizontal electrically conductive strip 606. Elements 600 that have no DC voltage bias applied lack piezoelectric properties and appear as electrical capacitors only. A primary challenge of this transducer array architecture for both imaging and therapeutic applications is the parallel capacitive loading that the non-activated elements have on the activated elements. The receive signal is loaded down by the apparent capacitance of the non-activated elements, thereby reducing the overall sensitivity of the array by however many elements are connected in parallel.

Accordingly, in one embodiment a PIN diode is used to form electrical connections only to the activated elements during the receive mode. The PIN diode allows only the activated elements in the 2D array to be electrically connected to the receive amplifiers, thereby reducing the parallel capacitive loading and allowing for sensitivities approaching that of 1D or 1.5D arrays. The connection may be “made” using only the electrical bias needed to activate the elements and therefore, does not require an additional actuation power distribution grid or electrical interconnects between elements.

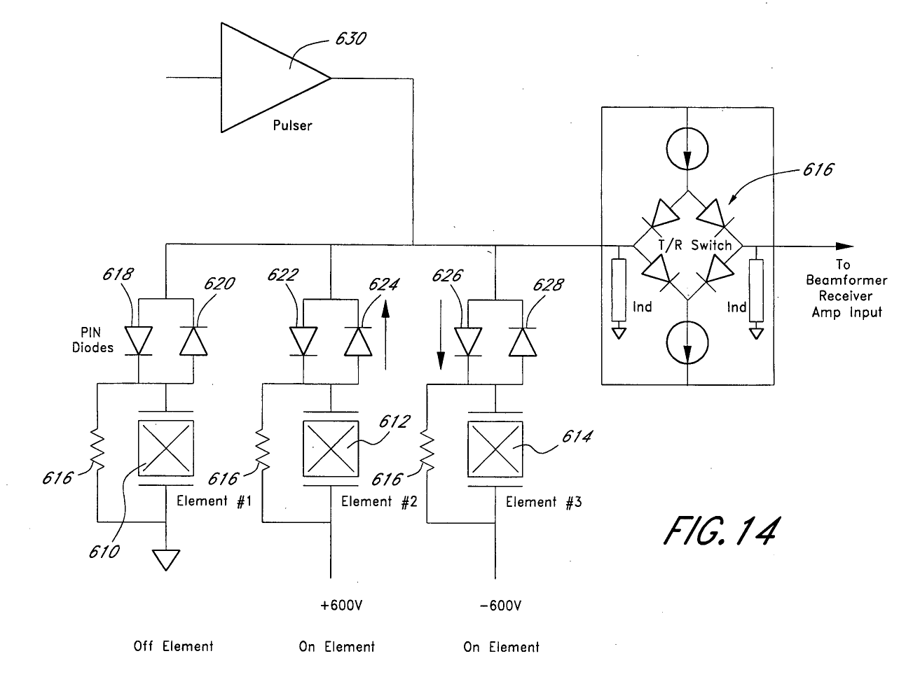

The use of a PIN diode as a selective switch in a 2D array of bias controllable piezoelectric material elements is illustrated by the electrical schematic in FIG. 14. In this circuit diagram, there are three elements 610, 612, and 614 connected in parallel to one beamformer receiver, through a T/R (Transmit/Receive) switch 616. Each array element 610, 612, and 614 has been enhanced with the addition of a DC current bias device 616 (depicted as a resistor) and a pair of PIN diodes 618, 620, 622, 624, 626, and 628. PIN diodes are not mandatory, but the ability of PIN diodes over normal diodes to conduct RF energy effectively when forward biased is a feature that can be exploited. Normal diodes would perform adequately, albeit at a reduced overall performance level.

In this example, the bottom electrode of element #1 610 is grounded (therefore non-activated) while elements #2 612 and #3 614 are biased to 600 volts, but at opposite potentials. In this described quiescent state (pulser 630 output has not been turned on), the diodes 618 and 620 connected to element #1 are not in a conductive state since there is no voltage across them. One diode 624 connected to element #2 would be conducting (shown with an arrow pointing in the direction of current flow) and would allow the top electrode to go to “one forward diode voltage drop” above ground. Also, one diode 626 connected to element #3 would be conducting and would allow the top electrode to go to “one forward diode voltage drop” below ground.

During the nominal pulse transmit period of the imaging mode or the therapeutic transmit mode, the output of the pulser 630 will have a voltage amplitude high enough to forward bias all the PIN diodes 618, 620, 622, 624, 626, and 628 in turn and electrically drive all three elements 610, 612, and 614. A pair of diodes is used because the pulser 630 drive output is bi-polar, going positive and negative. In FIG. 14, Element #1 610 is piezo-electrically inactive since there is no high voltage DC bias applied, causing Element #1 610 to just consume reactive electrical power and not contribute any acoustical output. Elements #2 and #3 612 and 614 are activated and will convert incoming electrical energy into acoustical output. The purpose of the T/R switch 616 is to shield the sensitive receive amplifier input from the pulser 630 drive output. The T/R switch 616 will mirror any small voltage on the left side input to the right side (e.g., voltages up to a maximum voltage of approximately +/−1 volt).

In receive mode (the period of time immediately following the cessation of the pulser output), the diodes return to the quiescent state described earlier. Returning acoustic echoes from the acoustical field will excite the elements 610, 612, and 614, causing small mV range signals to be produced on the activated elements. Since one of the PIN diodes connected to Element #2 612 is forward biased, the small signal generated on the top electrode (the bottom electrode is AC grounded by the 600 volt rail) will couple through the PIN diode 624 and go over to the left input of the T/R switch 616, to be mirrored over to the right side, for propagation into the receive amplifier. Likewise, one of the PIN diodes connected to Element #3 614 is forward biased, so the generated signal from that element will also make it to the receive amplifier. Element #1 610 is not connected to the circuit, since neither corresponding PIN diode 618 or 620 is biased on, nor does it load the signal line with extraneous intrinsic capacitance.

PIN diodes attached to the elements of a 2D array as described perform the function of automatically connecting and disconnecting elements as needed for optimum array performance. In FIG. 14 three elements are depicted, however, the technique can be used for an array of any size. Likewise, 600 volts is merely a representative DC bias voltage and any suitable bias voltage may be used. The circuit would also function as described at low voltages (e.g., with as little as 2 volts of bias) provided that electrostrictive material (e.g., PMN-PT) is used that can operate at those low electrical fields.

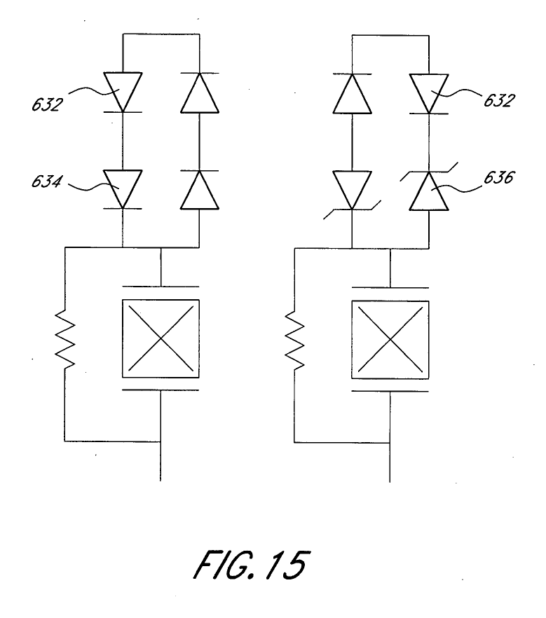

Finally, the above-described concept can also be utilized with other diode topologies that may provide different levels of performance. FIG. 15 illustrates electronic schematics of two other diode-element topologies. As these configurations illustrate, FIG. 15 use of two PIN diodes 632 and 634 in series or possibly a Zener diode 636 in series with a PIN diode 632, the parasitic capacitance of the off element is further removed from the activated elements. This may prove advantageous in very high count 2D arrays such as is used in the DBAC cuffs described above, where the non-activated elements could be as high as 300 elements. The use of two diodes 632 and 634 in series would also have the advantage of allowing for the receive signal to be over “one forward diode voltage drop” without accidentally turning on the diodes of a non-activated element. The Zener diode topology also blocks any pulser transmit output energy as long as that voltage of the output is less than the Zener breakdown voltage and the frequency of the transmit output is much higher than the RC time constant of the parasitic capacitance of the element and the effective resistance of the bias current element.

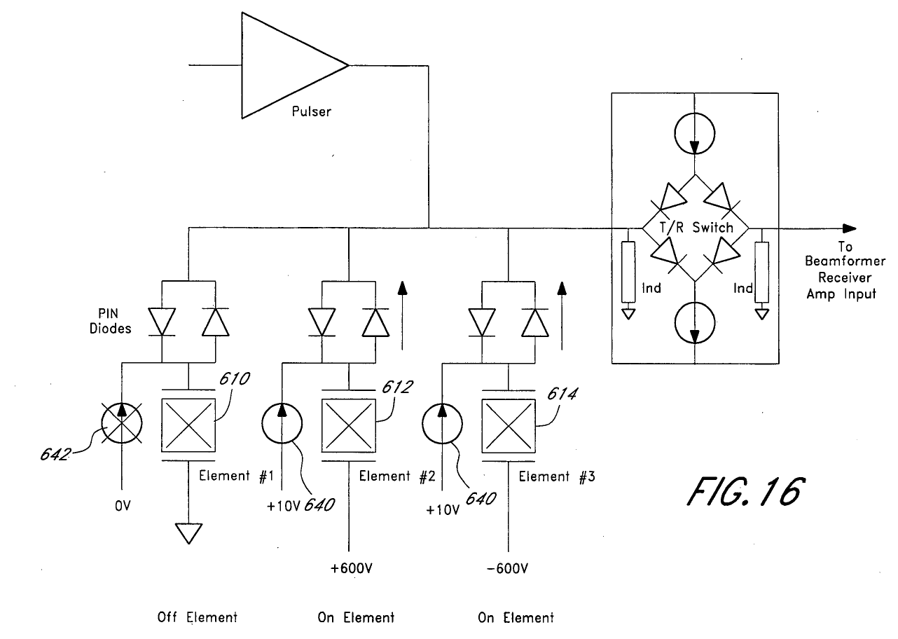

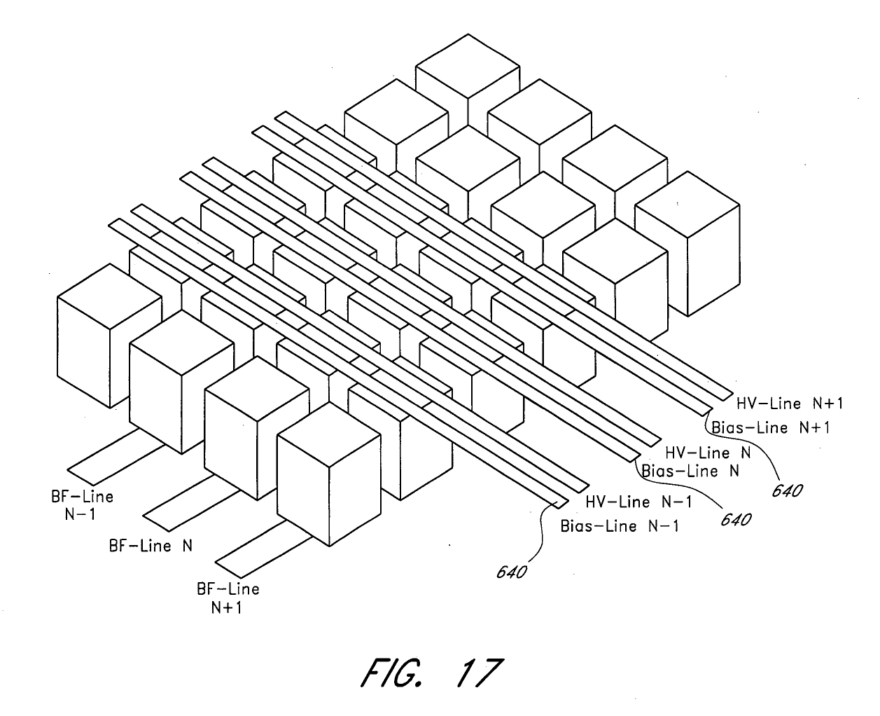

In yet another implementation, variation of the diode switch concept, the DC current bias device may be connected to a separate control/bias line rather than the high-voltage bias conducting strip. Such a configuration may provide improved switching speeds, improved crosstalk immunity between elements, or reduced power dissipation in the array. FIG. 16 is an electronic schematic showing the diode connection concept implemented with an extra +10 V voltage bias line 640. Although +10V has been illustrated, the actual voltage choice would be made based on design optimization input. The bias line would have little or no voltage if that column of elements were not selected for activation. In FIG. 16, the current source 642 connected to Element #1 610 is crossed out, to illustrate that the current source 642 does not have sufficient voltage to operate (i.e., is in an off state). The current sources 640 for Elements #2 and #3 612 and 614 are on since voltage is provided on their bias lines. The current sources supply current to the respective PIN diodes (current flow shown by arrows) and the elements re connected to the horizontal circuit line as explained earlier. The polarity of the diode bias current does not necessarily need to follow the high-voltage Bias polarity. FIG. 17 is a perspective view illustrating how an extra voltage bias line 640 may be added within a 2D array.

Use of PIN Diodes to Connect 2D Array Cross Points

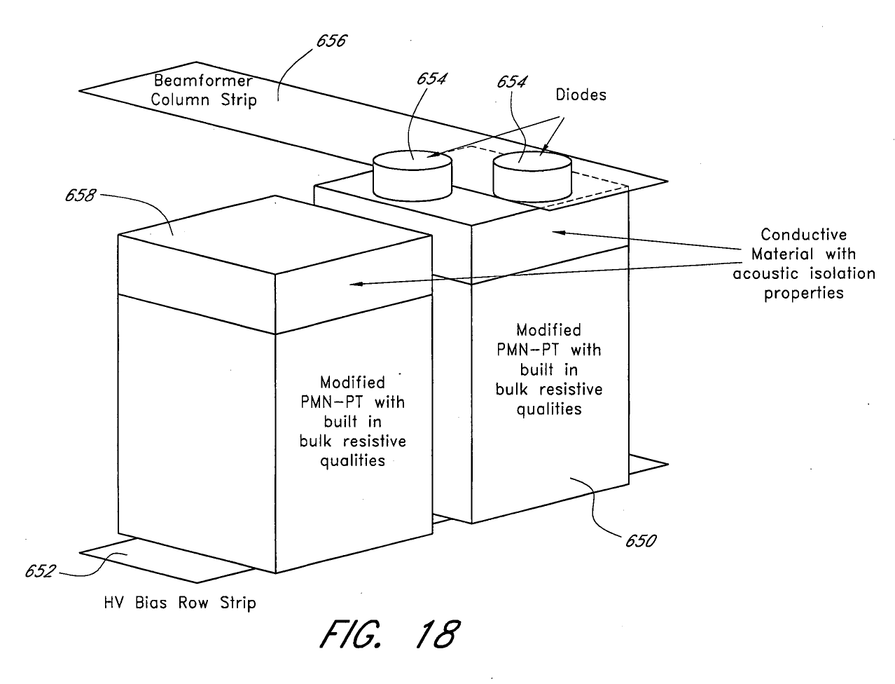

FIG. 18 is a perspective view of 2D array elements implemented with a modified PMN-PT material 650, formulated such that a small amount of DC leakage current flows through the element when the HV Bias Row Strip 652 is energized. This DC leakage current is enough to turn on the appropriate diode 654 above and electrically connect the element top electrode to the beamformer column strip 656. This configuration allows use of kerf saws to cut from below and above (following the direction of the conductive strips so as to not sever electrical connections) to generate the structures, thereby simplifying the manufacture of such an array (final kerf fill material and material surrounding the diodes and supporting the beamformer column strip 656 are not shown in FIG. 18). The diodes 654 may be strategically placed above the element and sized to be smaller than the surface area of the element. Thus, there is no physical limit to the number of array elements that can be arranged in this fashion. An acoustically isolating, electrically conductive material 658 can be used to hold the diodes 654 in place and at the same time better isolate the mass of the diodes 654 from the element, resulting in better image quality potential. In one embodiment, the acoustical isolating material is a carbon foam (e.g., POCO Foam™ obtainable from from POCO Graphite of Decauter, Tex.).

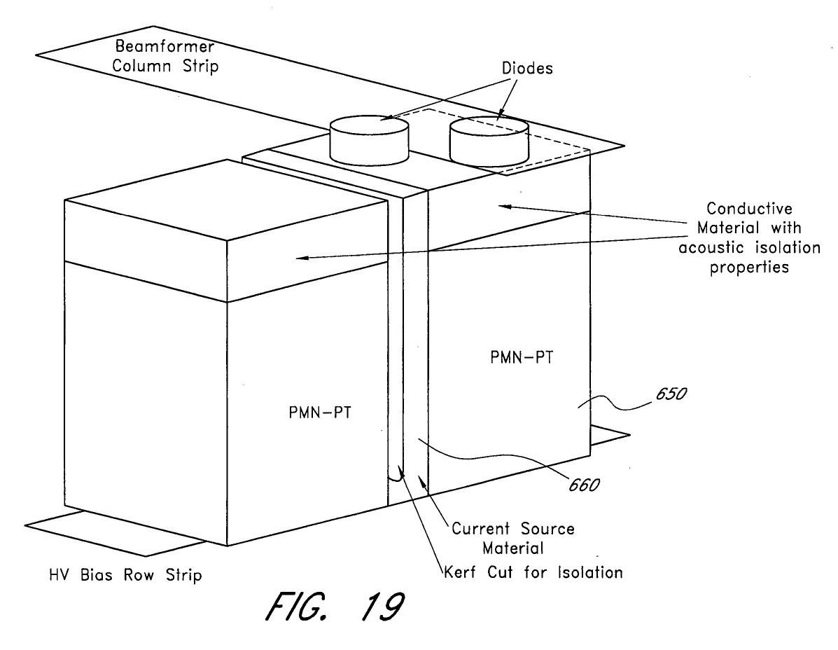

In another embodiment, conductive kerf fill 660 is inserted along one side of an element, and then subsequently cut to form the structure depicted by the perspective view of FIG. 19. This solution eliminates the need to modify the type of PMN-PT material 650 with DC conductive characteristics (as is the case for the embodiment illustrated in FIG. 18).

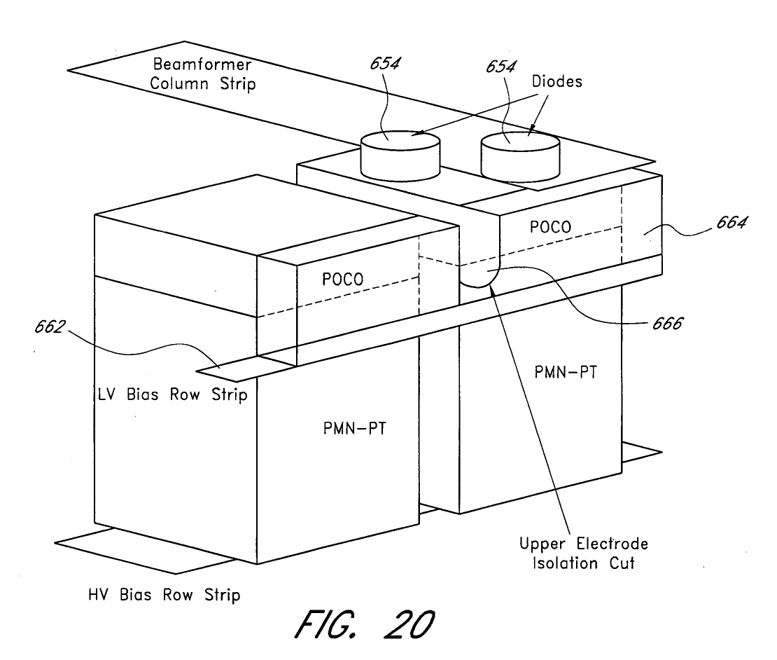

FIG. 20 is a perspective view illustrating yet another embodiment where the DC current to turn on the diode 654 comes from another strip, a “LV (Low Voltage) bias Row Strip” 662, implemented with conductive kerf fill 664 that is placed alongside the elements. An isolation cut 666 may be used to electrically disconnect the top electrodes from each other after assembly. This embodiment is an example of the PIN diode solution using an alternate means of sourcing the DC current (instead of taking it from the HV Bias).

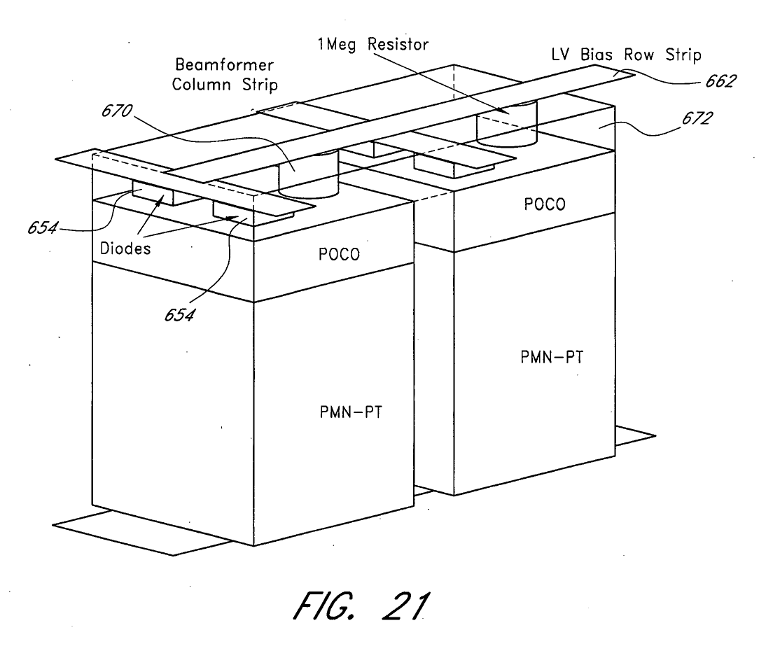

FIG. 21 is a perspective view illustrating an additional embodiment that uses two diodes 654 and a resistor 670 imbedded above each element. The diode 654 and resistor 670 provide the necessary circuitry to implement the PIN Diode solution. The diodes 654 and resistors 670 are “embedded” into a substrate 672. The substrate 672 could be silicon, Al Nitride, PCB FR4 materials, or any other suitable material. The substrate 672 allows for column and row direction conductive strips (e.g., the “LV Bias Row Strip” 662) to exist above the elements.

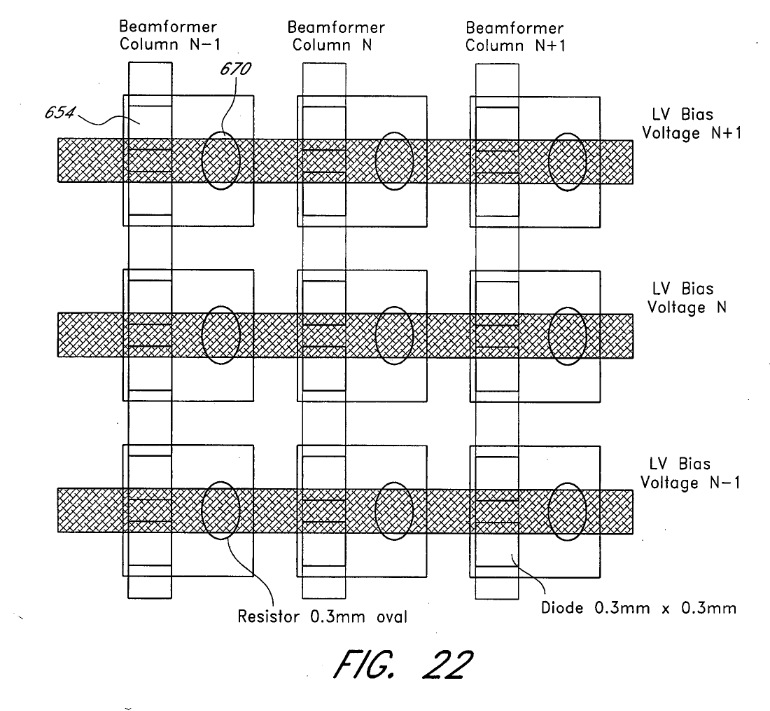

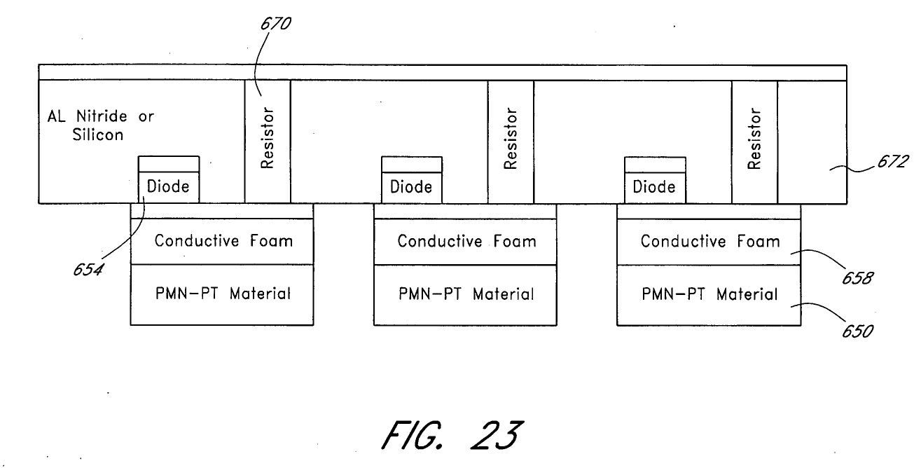

FIG. 22 is a top view and FIG. 23 is a cross-sectional view illustrating an alternative embodiment where the diode 654 and resistor 670 components are embedded into the substrate 672 first. The conductive acoustic isolation material 658 (e.g., a carbon foam) and PMN-PT layers 650 are then bonded on. This sequence of assembly allows for the top substrate 672 to support all kerf element cuts. The substrate 672 holds all the elements in place as they are being formed by the dicing saw. A solid substrate foundation allows for finer kerf cuts and smaller elements. Substrates such as Al nitride (and to a less degree, silicon) are good conductors of heat and can thus assist in the removal of unwanted heat. In one embodiment, a heat sink is bonded above the substrate 672 to further aid in heat dissipation. The substrate 672 will hold the diodes 654 and resistors 670 in place in the final array. In some embodiments, the substrate 672 serves as the original structure on which the components are built. Al nitride and nominal PCB fabrication techniques are industry standard and readily available to deploy toward this solution. In some embodiments, diode 654 and resistor 670 patterns of less than 1 mm pitch may be realized. Solid material on both sides of the acoustically isolating material layer 658 allows for the layer to be a softer foam material, such as a carbon foam.

Use of Electrostrictive Material in High Voltage Bypass Capacitors

Ultrasound transducers using PMN type electrostrictive ceramic materials typically require the use of a large, DC bias voltage to the element(s) for them to operate in the desired mode. Since the power supply for producing this bias voltage is usually placed in series with the element(s), it can have a detrimental effect on the AC impedance of the array as seen by the ultrasound transmitter and receiver. It is standard procedure to place a capacitor in parallel with the high voltage supply near the element(s) so that the AC signals bypass the DC supply, which effectively shorts out the impedance of the power supply and its interconnections from the perspective of the ultrasound transmitter and receivers.

In this application, the capacitor must be able to withstand a large DC bias voltage, which can be 100's to 1000's of volts. It also has a relatively large capacitance value, generally greater than 10,000 pF, to be effective at shorting the supply in the desired frequency range. This combination of requirements means that the capacitor is physically large in size using current state of the art manufacturing methods (e.g., a 10,000 pF, 1000 v, ceramic capacitor is typically a disc that measures 22 mm in diameter and 5 mm thick). For single element transducers, this large capacitor can be tolerated since only one capacitor is required. However, when building an array of elements, many bypass capacitors may be required because there are many individual elements in the array. As the number of capacitors increases, the amount of volume required for them can become impractical when a small, fine-pitched array is desired.

Accordingly, in some embodiments, the bypass capacitors are constructed using the same PMN material and construction methods as used in the transducer itself. Because of the unique properties of the PMN material and by constructing the capacitors along with the array, the volume used by the capacitors is thereby greatly reduced. Thus, an array of high voltage, high capacitance bypass capacitors can be constructed in a very small volume. This small volume makes the capacitor array practical for use with a 2 dimensional ultrasound transducer built from PMN material. This construction method also allows the capacitance values to be adjusted over a wide range without limitations of commercial, off-the-shelf availability. The value of the capacitors can be easily adjusted by varying the size of the plates during their manufacture.

In some embodiments, the capacitor array can be built in such a way that the interconnection of the capacitors to the transducer is relatively easy to accomplish using similar bond-wire interconnection techniques that are used for other connections on the transducer. In contrast, commercially available capacitors typically use interconnection methods that are more suited to printed circuit board assembly and are difficult to work with at the finer scale of an ultrasound transducer.

The capacitors can be constructed with a dielectric material that is identical to the material used in the transducer. Thus, the performance characteristics, such as temperature range, operating frequency and dielectric strength, of the capacitor will be matched to the transducer it is mated with. In contrast, commercial capacitors typically use ceramic dielectric formulations that are best suited for other applications, which may result in insufficient performance for certain specifications.

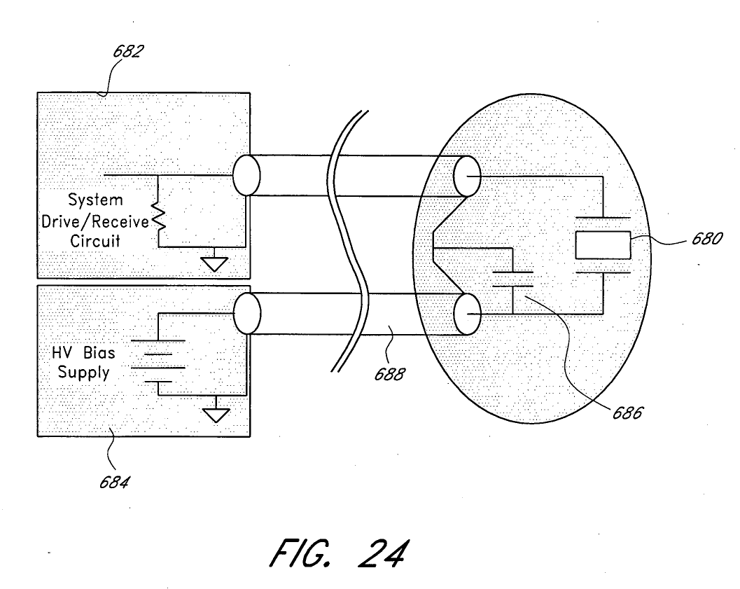

FIG. 24 is a circuit diagram illustrating a typical interconnection of a PMN transducer element 680 along with the system driving it 682 and the bias power supply 684. This figure illustrates that without the bypass capacitor 686 positioned near the element 680, the AC currents from the system must flow through the DC power supply 684 and its cabling 688. With the bypass capacitor 686, the AC currents can flow through the capacitor 686 directly between the system 682 and the piezoelectric transducer element 680. Typically, the bias voltage for PMN transducer elements 680 will be hundreds of volts, depending upon the thickness of the element. Thicker elements require higher voltage to get the desired field strength.

It has been discovered that a 0.5 mm thick element improves in performance as the voltage is increased. A bias voltage of 400 V DC appears to be near the “knee of the curve” such that below that voltage, the performance is poor. Above 400 V the efficiency of the acoustic output continues to increase but at a slower rate than below 400 V. Thus, for a 0.5 mm element, 400 V is the preferable minimum usable bias voltage. When thicker elements are used, which may be desired in order to achieve resonance close to 1 MHz, a proportionally higher bias voltage can be used. Thus, a typical minimum value for voltage tolerance on the bypass capacitor is 1000 V or more. As noted above, the current state of the art in off-the-shelf capacitors of this rating provide a maximum capacitance of 0.01 uF in a disk that is 22 mm diameter by 5 mm thick. Custom parts or more exotic materials may produce smaller or higher capacitance, but cost and delivery times go up substantially.

An ideal bypass capacitor would exhibit near zero impedance at the frequency of interest or at least be substantially lower in impedance than the piezoelectric element and substantially lower impedance than the cable and power supply it is bypassing. At a nominal frequency of 1.5 MHz, which is approximately where a therapeutic transducer might be designed to operate, the 0.01 uF capacitor will have an impedance of 10.6 ohms. This is probably higher than would be desired, which means even the largest available capacitor in the 1 kV rating is less than ideal.

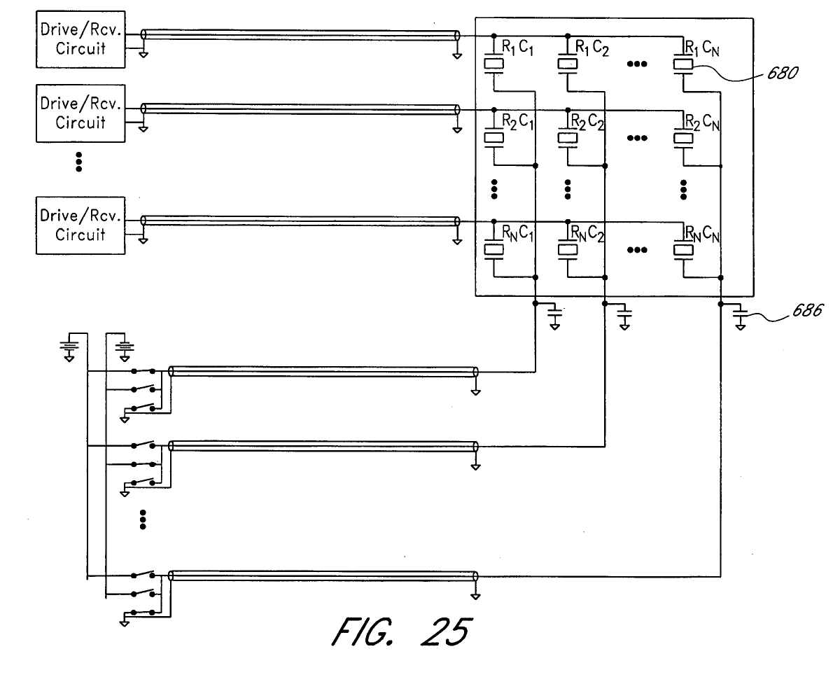

FIG. 25 is a circuit diagram illustrating how an array of PMN elements 680 can be connected in a row and column fashion. In this example, a separate bypass capacitor 686 is used for each column in the array. For example if a 20×20 array were constructed with each element 680 being 1 mm square, then 20 bypass capacitors 686 would be needed for a 2 cm square array. Using the off-the-shelf capacitors mentioned above, a set of 20 of these capacitors would require a cylinder 2.2 cm in diameter by 10 cm tall.

As an alternative, consider the equation for the capacitance of a device:
C=Εr*8.85 pF/meter*A/D

where:
C=capacitance
Εr=relative dielectric constant of the insulator
A=area of the plates
D=distance between the plates (thickness of the dielectric)

One of the useful properties of PMN ceramic is its very high dielectric constant. The exact value varies with formulation, temperature and bias level, but 15,000 is a reasonable average value. By plating both sides of a 1 cm square wafer of PMN that is 0.5 mm thick, a capacitor of 0.026 uF is produced. Thus, a simple plated wafer of the same material used for a transducer can produce a capacitor that is 2.5 times as large as the commercial version in a fraction of the space. Another property of PMN is its high dielectric strength, which is specified as having a working range up to 10 Kv/cm and will withstand values well beyond that. The thickness of the PMN can be tailored as can the area of the plating to optimize the capacitance and the voltage rating. Allowing for some space on the sides of the wafer to attach bond wires, insulation, and space between them, the wafers can be placed side-by-side on a pitch of 2 mm. The 20 capacitors required for the example array described above would then require a cube that is 1 cm×1 cm×4 cm. Or alternatively a cube that is 1 cm×2 cm×2 cm.

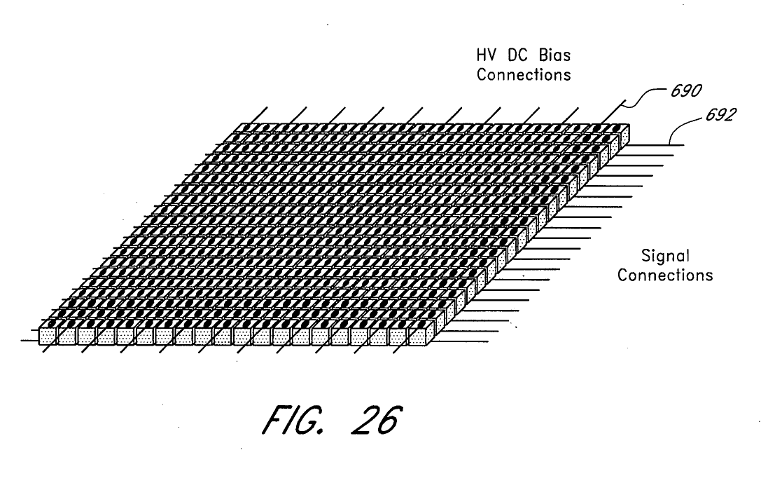

FIG. 26 is a perspective view illustrating a capacitor stack in combination with a transducer array in a 20×20 array (any size array is possible). This array has approximate dimensions of 2 cm on a side and only 1 or 2 mm thick. The bias connections 690 run in one direction across the array's columns. The signal drivers 692 run in the orthogonal direction across the rows. The pitch of the bias columns 690 is 1 mm and the connections could be attached at either side. If every other column is terminated on opposite sides of the array, the pitch of the terminations would be 2 mm. Given the high voltage that is being dealt with, the wider pitch of this approach has definite advantages compared with connecting all 20 columns on one side of the array.

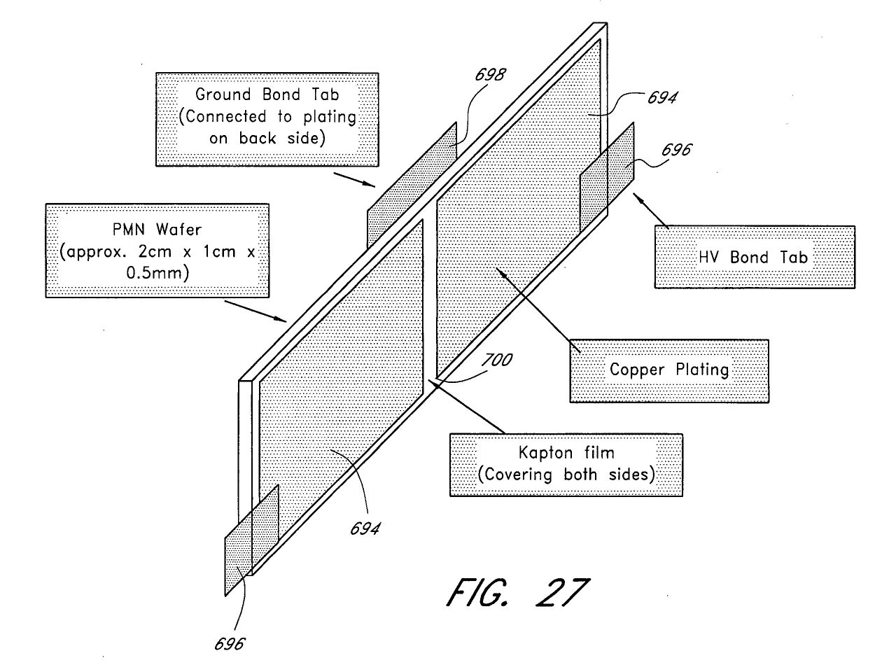

The capacitors may be built from 1 cm×2 cm×0.5 mm wafers of PMN material. Each wafer is copper plated on both sides. One side is used for ground and is fully plated. The other side is used for the DC voltage connections and the plating is split in the middle into two separate plates. In this configuration, each wafer would make two capacitors that are approximately 1 cm×1 cm each. To prevent acoustic coupling between the two capacitors, the PMN material may be completely split and then kerf-filled between the two halves. FIG. 27 is a perspective view illustrating FIG. 27 the two plates 694 on the HV side of the wafer having a small copper foil 696 bonded to each. The foil 696 extends out the side for attachment to the transducer array. The ground side of the capacitors also has copper foil 698 bonded to it, which can be connected to system ground. Each side of the wafer is covered with a thin insulating film 700, such as Kapton. The dimensions of the wafers that are described here (1 cm×2 cm) are not critical. The 2 cm dimension can be adjusted as necessary to create the best fit over the transducer array. The 1 cm dimension can be adjusted up or down as necessary to vary the amount of capacitance that is produced.

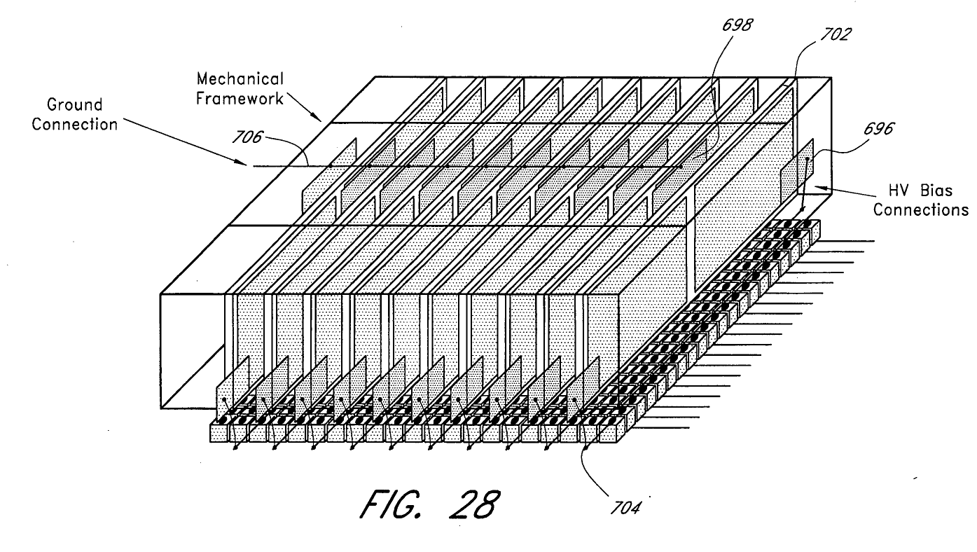

To assemble the capacitor array, a mechanical framework can be built that holds ten of the 1 cm×2 cm capacitor assemblies side by side as shown in the perspective view of FIG. 28. Placing the wafers 702 on 2 mm pitch allows room for the bonding foil 696 and 698, the insulating foil, and some air gap between them. Because the capacitors are built of a piezoelectric material (PMN) and because they are biased, they will vibrate when AC current is run through them. To prevent acoustic crosstalk between the adjacent capacitors, they are separated by a material that is an attenuator to the acoustic energy in the 1+MHz range. A small air gap should be adequate for this attenuation. With the plates on a 2 mm pitch, ten plates span 2 cm and fit over the 2 cm×2 cm transducer. This configuration allows for aligning the bonding foil 696 for the HV side of the capacitors over the HV connections 704 on the sides of the transducer array. Various methods from simple bond wires to more elaborate connectors can be used to attach the HV connections 704 on the capacitor array to the terminations on the transducer array. The ground connections 706 from each wafer that extend out the top of the array can be connected together and then connected to the return lines for the transducer's I/O cables.

When assembling the capacitor wafers into an array, care must be taken to prevent electrical breakdown due to the high voltages that are being handled. The gap between the two HV plates on each wafer may be made wide enough for electrical isolation and covered with a good dielectric. This gap may experience twice the voltage differential of the high voltage supply since one side may have a positive bias and the other may be negative. The edges of the wafers can be covered with a strong dielectric material to prevent arcing across the edge of the PMN. The bonding foil and the connections to the transducer may also be adequately insulated from one another to prevent arcing across them.

Use of Non-activated Elements as Intrinsic High Voltage Bypass Capacitors

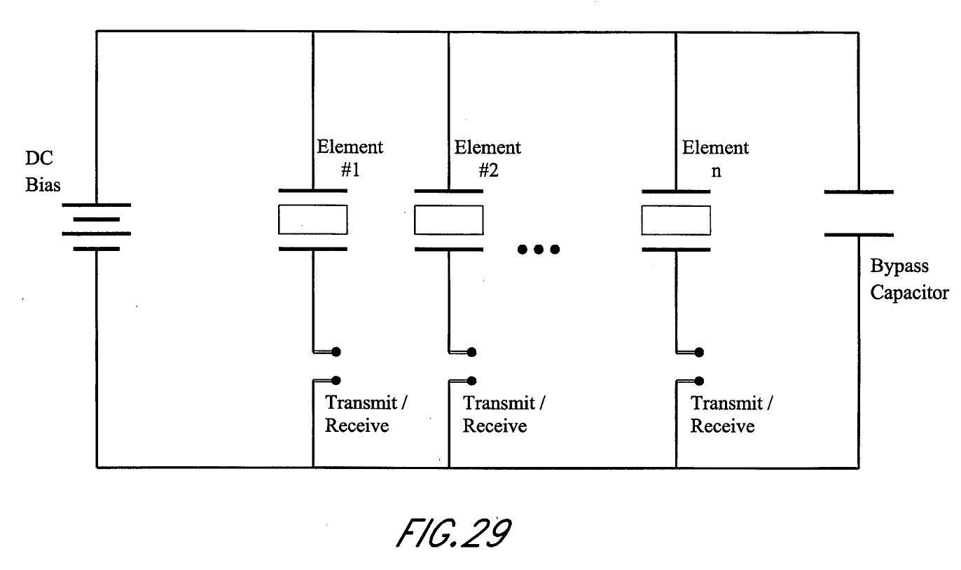

As mentioned in the previous section, an important design consideration of bias controlled 2D arrays is the need to have a low impedance ground path for all the elements along a common high-voltage strip, normally provided by the addition of a bypass capacitor in the circuit. An example of this typical bypass solution is depicted in FIG. 29. The purpose of the bypass capacitor is to limit the electrical crosstalk between elements.

In one embodiment, non-activated elements of a 2D array are used to provide the bypass capacitance, eliminating or reducing the size of additional bypass capacitors. As discussed above, a given element in a electrostrictive transducer array is only activated if a DC high-voltage bias is applied by the electrically conductive strip above and simultaneously, an AC drive signal from below. Elements which have no DC bias applied appear as electrical capacitors only and lack any acoustic-electric transforming properties. By extension, it is possible to select an active acoustic “aperture”, which is the set of all elements that have a DC high-voltage bias applied and are also connected to an AC drive Transmit/Receive circuit. In these configurations, the added bypass capacitor needed for optimal detection (e.g. imaging) performance can be reduced, possibly to the point of not being required at all. This is significant because the bypass capacitor required (high voltage and fairly high capacitance) is physically large, which could limit certain useful applications (e.g., the DBAC cuff described above).

In a large 2D array of elements, where there are more elements than the nominal sized active aperture, there will usually be elements that are not activated. For bias controllable piezoelectric materials (such as PMN-PT) and potentially other bias controlled MEM structures (such as C-MUT or P-MUT), these non-activated elements still possess electrically capacitive properties, even though their electro-acoustic characteristics have not been switched on.

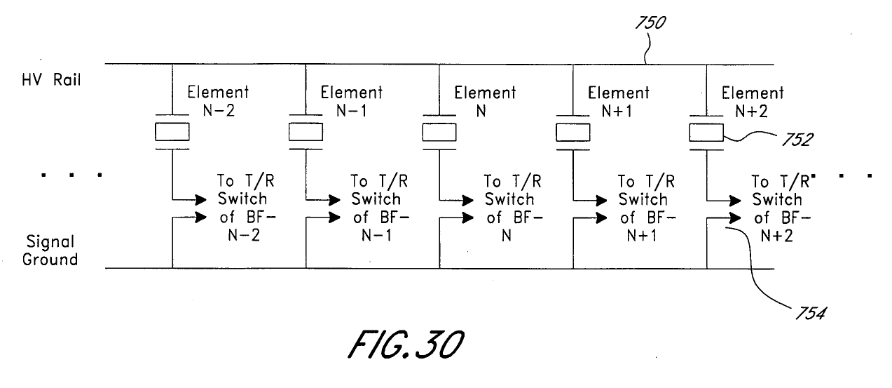

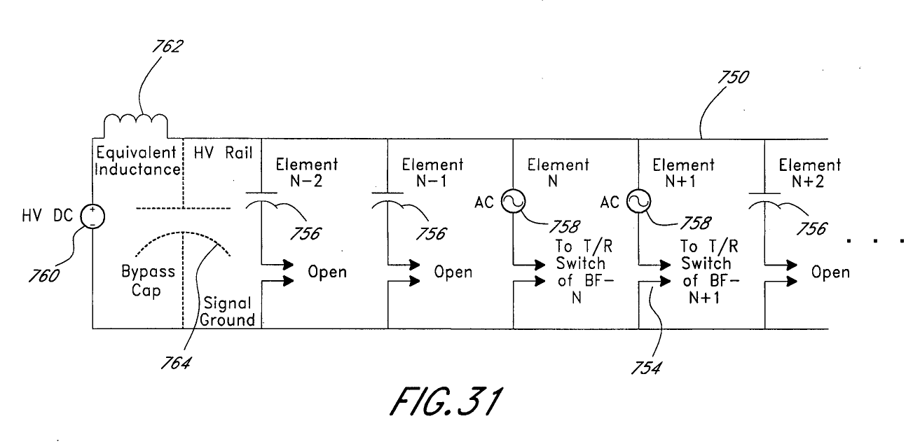

FIG. 30 is a circuit diagram illustrating a sample high voltage rail 750 with the elements 752 connected thereto and on the other side, connected to a corresponding T/R switch 754, and ultimately to a beamformer channel (receive amplifier). For the sake of discussion, it is assumed that only “element N” and “element N+1” are part of the desired active aperture for this excitation interval. Thus, T/R switch BF-N and T/R switch BF−N+1 would be enabled. All other T/R switches would be disconnected, as is typical in an imaging system. The circuit diagram would reduce to the circuit shown in FIG. 31.

In FIG. 31, the unconnected elements are shown as capacitors 756. Element N and element N+1 are shown as AC generators 758, since they are connected to the T/R switch 754 and hence will react to any acoustic pressure by producing an AC signal. Added to the circuit diagram is a HV DC power source 760, separated by an inductor 762, to model the fact that the power supply 760 is some physical distance away from the 2D array. The previously mentioned bypass capacitor 764 is also shown, placed on the element side of the inductor 762, as it should be in order to hold the HV rail 750 voltage stable. The activated elements act like independent AC generators 758 (in the response to dissimilar acoustic pressure) that cause the voltage at the voltage rail 750 to fluctuate asynchronously and cause electrical crosstalk.

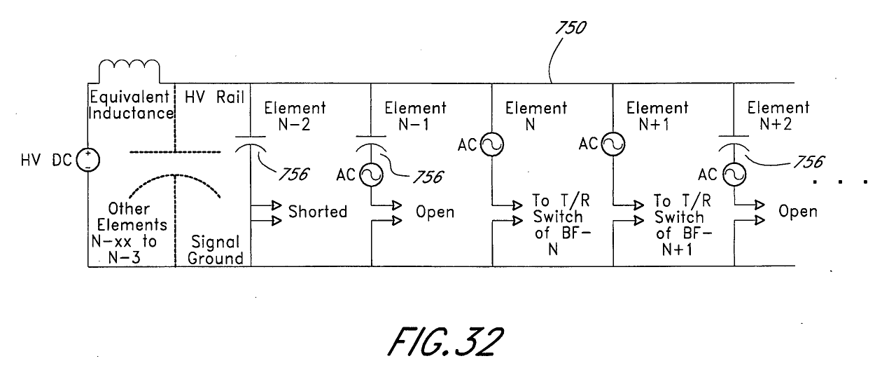

In some embodiments, certain elements may be grounded instead of being left open. The circuit diagram then would reduce to the slightly different schematic depicted in FIG. 32. In this new circuit diagram, elements 756 not part of the active aperture along the HV rail 750 are intelligently selected to be shorted or left open. The selection criterion may be based on an estimate of whether or not the element 756 would receive significant acoustic pressure. Elements which would receive acoustic pressure (elements N−1 and N+2 in this example) may be left open so as to not allow them to become part of the circuit. These elements are modeled in the circuit diagram as both a capacitor and an AC generator to emphasize the fact that they are capacitors when not connected to a T/R switch, but would become AC “noise” generators if shorted to ground. Elements N-xx to N−2 are outside the acoustic field and are shorted so as to deploy them as bypass capacitors. In the case where the 2D array is in transmit mode, all the elements outside the desired active aperture are shorted to ground. In this mode, the “noise” generator characteristic of the outside elements is not a consideration and the full advantage of the extra intrinsic capacitance available should be utilized.

Although the invention has been described with reference to embodiments and examples, it should be understood that numerous and various modifications can be made without departing from the spirit of the invention. Accordingly, the invention is limited only by the following claims.

What is claimed is:

1. An ultrasound cuff, comprising:
an array of ultrasound transducers adapted to be deployed circumferentially around a body limb; and
a pressurizeable bladder positioned on a first side of the array, wherein the cuff is configured such that when the array is deployed circumferentially around the body limb, the bladder is positioned between the array and the body limb.

2. The cuff of claim 1, where the ultrasound transducers are adapted for both therapy and detection.

3. The cuff of claim 1, comprising a substantially non-expandable layer positioned on a second side of the array opposite the first side, wherein the non-expandable layer is configured to prevent the flexible array from expanding outward when the cuff is deployed around the body limb and the bladder is pressurized.

4. The cuff of claim 1, comprising a plurality of the pressurizeable bladders.

5. The cuff of claim 1, comprising a liquid pressurization module configured to pressurize liquid disposed within the bladder.

6. The cuff of claim 5, wherein the pressurization module is configured to circulate liquid through the bladder.

7. The cuff of claim 5, wherein the pressurization module is configured to cool the liquid.

8. The cuff of claim 5, wherein the pressurization module is configured to degas the liquid.

9. The cuff of claim 1, wherein the ultrasound transducers are configured to provide ultrasonic energy with enough intensity to induce hemostasis.

10. The cuff of claim 1, wherein the bladder covers the entire array.

11. The cuff of claim 1, wherein the ultrasound transducers comprise electrostrictive material.

12. The cuff of claim 11, wherein the electrostrictive material is lead-magnesium-niobate modified with lead titanate.

13. The cuff of claim 1, wherein the ultrasound transducers comprise PZT material.

14. An ultrasound cuff, comprising:
an array of ultrasound transducers having an approximately cylindrical shape; and
a bladder filed with a liquid, the bladder covering the surface of the transducers that are interior to the cylindrical shape.

15. The cuff of claim 14, wherein the liquid is water.

16. The cuff of claim 14, wherein the liquid is physiological saline.

17. The cuff of claim 14, comprising an acoustic couplant material between the bladder and the transducers.

18. The cuff of claim 17, wherein the acoustic couplant material comprises a gel.

19. The cuff of claim 14, comprising an acoustic couplant material disposed on the surface of the bladder that is interior to the cylindrical shape.

20. The cuff of claim 14, wherein the ultrasound transducers comprise electrostrictive material.

21. The cuff of claim 20, wherein the electrostrictive material is lead-magnesium-niobate modified with lead titanate.

22. The cuff of claim 14, wherein the ultrasound transducers comprise PZT material.

23. An ultrasound cuff, comprising:
a flexible array of ultrasound transducers adapted to be deployed circumferentially around a body limb;
at least one seal positioned along at least one edge of at least some of the ultrasound transducers, the seal configured to contact the body limb when the array is deployed circumferentially around the body limb such that a space is maintained between the body limb and the at least some transducers.

24. The cuff of claim 23, wherein the seal comprises a pressurizeable bladder configured to maintain said space when the bladder is pressurized.

25. The cuff of claim 24, comprising a pressurization module configured to pressurize the first pressurizeable bladder.

26. The cuff of claim 25, wherein the pressurization module is a gas pressurization module.

27. The cuff of claim 23, wherein the seal comprises silicone or foam pliant material.

28. The cuff of claim 23, wherein the array comprises a plurality of ultrasound transducer panels, wherein one of said seals extends around the perimeter of each transducer panel.

29. The cuff of claim 23, comprising two of said seals, a first seal positioned on a first side of the array and a second seal positioned on a second side of the array.

30. The cuff of claim 29, wherein the first seal comprises a first pressurizeable bladder configured such that when the array is deployed circumferentially around the body limb, the first bladder also extends circumferentially around the body limb and wherein the second seal comprises a second pressurizeable bladder configured such that when the array is deployed circumferentially around the body limb, the second bladder also extends circumferentially around the body limb.

31. The cuff of claim 30, wherein when the array is deployed circumferentially around the body limb and the first and second pressurizeable bladders are pressurized, the first and second pressurizeable bladders are configured to maintain said space between the body limb and the array.

32. The cuff of claim 23, comprising an inlet port configured to facilitate the introduction of a liquid into the space.

33. The cuff of claim 23, comprising a liquid pressurization module configured pressurize liquid disposed within the space.

34. The cuff of claim 23, wherein the ultrasound transducers comprise electrostrictive material.

35. The cuff of claim 34, wherein the electrostrictive material is lead-magnesium-niobate modified with lead titanate.

36. The cuff of claim 23, wherein the ultrasound transducers comprise PZT material.

37. A method of effecting hemostasis in a wound in a limb, the method comprising:
placing an inflatable cuff with integrated ultrasound transducers around the limb;
inflating the cuff; and
applying high intensity focused ultrasound sufficient to effect hemostasis with the transducers.

38. The method of claim 37, wherein the cuff is inflated to a pressure sufficient to reduce intravascular blood flow.

39. The method of claim 37, wherein the cuff is inflated to a pressure sufficient to stop intravascular blood flow.

40. The method of claim 37, wherein the cuff is inflated to a pressure sufficient to reduce extravascular blood flow.

41. The method of claim 37, wherein the cuff is inflated to a pressure sufficient to stop extravascular blood flow.

42. The method of claim 37, wherein inflating the cuff comprises introducing a liquid into a bladder in the cuff.

43. The method of claim 37, wherein the cuff is inflated to a pressure sufficient to splint the limb.

44. The method of claim 42, wherein the bladder is positioned between the limb and the transducers.

45. The method of claim 42, comprising cooling the liquid while applying the high intensity focused ultrasound.

46. The method of claim 37, wherein inflating the cuff comprises inflating a first and second bladder, wherein the inflated bladders define a space between the limb and the transducers.

47. The method of claim 46, comprising introducing a liquid into the space.

48. The method of claim 47, wherein the liquid comprises a pro-coagulant agent.

49. The method of claim 48, wherein the pro-coagulant is thrombin.

50. The method of claim 47, wherein the liquid comprises an anti-infection agent.

51. The method of claim 50, wherein the anti-infection additive is an anti-biotic.

52. A method of effecting hemostasis in a wound in a limb, the method comprising:
placing a tourniquet on the limb;
placing a cuff with integrated ultrasound transducers around the limb;
applying high intensity focused ultrasound sufficient to effect hemostasis with the transducers.

53. An ultrasound applicator patch, comprising:
an array of ultrasound transducers;
sidewalls disposed around the perimeter of the array, wherein the sidewalls define a space over the ultrasound transducers; and
an inlet port in the sidewalls or the array configured to allow introduction of a fluid into the space.

54. The patch of claim 53, comprising an adhesive disposed on the sidewalls.

55. The patch of claim 53, comprising a strap coupled to the transducers and configure to encircle the limb.

56. A method of effecting hemostasis in a wound, comprising:
positioning the patch of claim 53 over the wound;
introducing a fluid through the inlet port into the space; and
applying high intensity focussed ultrasound sufficient to effect hemostasis with the transducers.

57. An ultrasound applicator patch, comprising:
an array of ultrasound transducers; and
a pressurizeable bladder positioned on a first side of the array, wherein the cuff is configured such that when the array is deployed on the wound, the bladder is positioned between the array and the wound.

58. The patch of claim 57, comprising a strap coupled to the transducers and configure to encircle the limb.

59. A method of effecting hemostasis in a wound, comprising:
positioning the patch of claim 57 over the wound;
introducing a fluid into the pressurizeable bladder; and
applying high intensity focused ultrasound sufficient to effect hemostasis with the transducers.
